(19) United States
(12) Patent Application Publication
(10) Pub. No.: US 2026/0077030 A1
(43) Pub. Date: Mar. 19, 2026

(54) MEMBRANE VESICLES COMPRISING MULTIPLE INFLUENZA ANTIGENS FOR VACCINES

(71) Applicant: VERSATOPE THERAPEUTICS, INC., Lowell, MA (US)

(72) Inventors: Christopher LOCHER, Lexington, MA (US); Kien T. NGUYEN, Rockville, MD (US); Carlos H. FAERMAN, Hudson, MA (US)

(21) Appl. No.: 19/331,556

(22) Filed: Sep. 17, 2025

Related U.S. Application Data
(63) Continuation of application No. PCT/US2024/020044, filed on Mar. 15, 2024.
(60) Provisional application No. 63/491,005, filed on Mar. 17, 2023.

(51) Int. Cl. A61K 39/145; A61K 39/00; A61P 37/04; C07K 14/005

(52) U.S. Cl. CPC A61K 39/145; A61P 37/04; C07K 14/005; A61K 2039/6093; A61K 2039/70; C12N 2760/16122; C12N 2760/16134; C12N 2760/16171

(57) ABSTRACT

Described herein, are multivalent influenza virus vaccines (MIV), multivalent immunogenic polypeptides (MIP), and fusion proteins. Provided herein are further methods of making and using the MIVs, MIPs, and fusion proteins.

### CROSS-REFERENCE

This application is a continuation of International Application No. PCT/US2024/020044, which designed the United States and was filed on Mar. 15, 2024, published in English, which claims the benefit of U.S. Provisional Patent Application No. 63/491,005, filed Mar. 17, 2023. The entire teachings of the above-referenced applications are incorporated herein by reference.

### SEQUENCE LISTING

The sequence listing submitted via EFS, in compliance with 37 CFR § 1.52(e)(5), is incorporated herein by reference. The sequence listing XML file submitted via EFS contains the file “4377.1003 US.xml”, created on Mar. 14, 2024, which is 25,040 bytes in size.

### SUMMARY OF THE DISCLOSURE

In some aspects of the disclosure, a multivalent influenza virus vaccine (MIV) is provided, said MIV comprising: A-B-C; wherein A is a transport protein or a fragment thereof comprising at least domains X₁ and X₂, wherein X₁ is a transmembrane domain polypeptide, and X₂ is a polypeptide capable of being operably linked to B; B is a multivalent immunogenic polypeptide (MIP) comprising at least five influenza virus immunogenic epitopes selected from the group consisting of Y₁, Y₂, Y₃, Y₄, Y₅, and Y₆, C is a membrane vesicle derived from a genetically modified gram positive bacteria; wherein A and B are operably linked, resulting in a multivalent immunogenic transmembrane polypeptide; wherein A-B is linked to C via the transmembrane domain X₁, thereby forming the MIV; and wherein the MIV is capable of eliciting an immune response to at least two influenza strains when administered to a mammal.

In some aspects of the disclosure, a multivalent influenza virus vaccine (MIV), is provided, said MIV comprising: a transport protein or a fragment thereof, wherein said transport protein comprises a transmembrane domain; a multivalent immunogenic polypeptide (MIP) comprising at least five covalently linked influenza virus immunogenic epitopes; a membrane vesicle derived from a genetically modified gram positive bacteria; wherein the transport protein of a) is operably linked to the MIP of b), resulting in a multivalent immunogenic transmembrane polypeptide; wherein the multivalent immunogenic transmembrane polypeptide is linked to the vesicle forming the MIV; and wherein the MIV is capable of eliciting an immune response to at least 2 influenza virus strains when administered to a mammal.

In some embodiments, the transport protein is an adhesin, immunomodulatory compound, protease, or toxin, or a fragment thereof. In some embodiments, the transport protein is ClyA. In some embodiments, the ClyA comprises an amino acid sequence at least about 80% identical to SEQ ID NO: 1.

In some embodiments, the at least five influenza virus immunogenic epitopes are fused in tandem. In some embodiments, the operable linkage between A and B comprises a covalent linkage. In some embodiments, the at least five influenza virus immunogenic epitopes are fused to a N- or C-terminus of the transport protein. In some embodiments, the at least five influenza virus immunogenic epitopes are presented on outside of the vesicle.

In some embodiments, the influenza virus is influenza A. In some embodiments, the influenza A is human, swine, or avian. In some embodiments, the avian is a chicken, a whooper a swan, a quail, or a mallard. In some embodiments, the influenza A is influenza subtype is H1, H2, H3, H5, H6, H7, or H9. In some embodiments, the influenza virus is selected from the group consisting of H1N1, H1N2, H2N1, H3N2, H5N1, H5N2, H9N2, H7N9, H7N7, H7N3, H6N6, H6N2, and H6N1.

In some embodiments, the at least five influenza virus immunogenic epitopes are different. In some embodiments, the at least five influenza virus immunogenic epitopes are the same. In some embodiments, at least one of the at least five influenza virus immunogenic epitopes comprises a consensus sequence or a non-naturally occurring sequence.

In some embodiments, the MIP comprises an influenza A matrix protein 2 extracellular (M2e) peptide or fragment thereof. In some embodiments, the MIP comprises five M2e peptides. In some embodiments, the MIP comprises six M2e peptides. In some embodiments, the M2e peptide comprises the amino acid sequence at least about 90% identical to any one of SEQ ID NOs: 2-8. In some embodiments, the transport protein and the MIP are linked using one or more linkers.

In some embodiments, the one or more linkers comprises a sequence selected from the group consisting of (GS)n (SEQ ID NO: 12), (G2S)n (SEQ ID NO: 13), (G3S)n (SEQ ID NO: 14), (G4S)n (SEQ ID NO: 15), and (G)n (SEQ ID NO: 16), and wherein n is an integer from 2 to 20. In some embodiments, the one or more linkers comprises a sequence selected from the group consisting of (GGSGGD)n (SEQ ID NO: 17) or (GGSGGE)n (SEQ ID NO: 18), and wherein n is an integer from 2 to 6. In some embodiments, the one or more linkers comprises a sequence selected from the group consisting of (GGGSGGG)n (SEQ ID NO: 19), (GGGSGSGGGGS)n (SEQ ID NO: 20) and (GGGGGPGGGGP)n (SEQ ID NO: 21), and wherein n is an integer from 1 to 3. In some embodiments, the one or more linkers comprises a sequence selected from the group consisting of (GX)n, (GGX)n, (GGGX)n, (GGGGX)n, and (GzX)n, wherein z is between 1 and 20, and wherein n is at least 8. In some embodiments, X is serine, aspartic acid, glutamic acid, threonine, or proline.

In some embodiments, Y₁, Y₂, Y₃, Y₄, Y₅, and Y₆ are selected from the group consisting of a human M2e peptide, a swine M2e peptide, a swan M2e peptide, a chicken M2e peptide, and a mallard M2e peptide. In some embodiments, the multivalent immunogenic transmembrane polypeptide comprises the amino acid sequence having a formula ClyA-(M2e)₅. In some embodiments, the multivalent immunogenic transmembrane polypeptide comprises the amino acid sequence having a formula ClyA-(M2e)₆. In some embodiments, the multivalent immunogenic transmembrane polypeptide comprises the amino acid sequence having a formula ClyA-(M2e-1)-(M2e-2)-(M2e-3)-(M2e-4)-(M2e-5)-(M2e-6), wherein M2e-1 comprises human M2e peptide, wherein M2e-2 comprises swine M2e peptide, wherein M2e-3 comprises swan M2e peptide, wherein M2e-4 comprises chicken M2e peptide, wherein M2e-5 comprises chicken M2e peptide, and wherein M2e-6 comprises mallard M2e peptide. In some embodiments, the multivalent immunogenic transmembrane polypeptide comprises the amino acid sequence having a formula ClyA-(M2e-1)-(M2e-2)-(M2e-3)-(M2e-4)-(M2e-5)-(M2e-6), wherein M2e-1 comprises the amino acid sequence at least about 90% identical to SEQ ID NO: 3, wherein M2e-2 comprises the amino acid sequence at least about 90% identical to SEQ ID NO: 4, wherein M2e-3 comprises the amino acid sequence at least about 90% identical to SEQ ID NO: 5, wherein M2e-4 comprises the amino acid sequence at least about 90% identical to SEQ ID NO: 6, wherein M2e-5 comprises the amino acid sequence at least about 90% identical to SEQ ID NO: 7, and wherein M2e-6 comprises the amino acid sequence at least about 90% identical to SEQ ID NO: 8. In some embodiments, the multivalent immunogenic transmembrane polypeptide comprises the amino acid sequence at least about 80% identical to SEQ ID NO: 9.

In some embodiments, the gram positive bacteria is selected from the group consisting of: Bifidobacterium longum, Bifidobacterium lactis, Bifidobacterium animalis, Bifidobacterium breve, Bifidobacterium infantis, Bifidobacterium adolescentis, Lactobacillus acidophilus, Lactobacillus casei, Lactobacillus paracasei, Lactobacillus salivarius, Lactobacillus reuteri, Lactobacillus rhamnosus, Lactobacillus johnsonii, Lactobacillus plantarum, Lactobacillus fermentum, Lactococcus lactis, Streptococcus thermophilus, Lactococcus lactis, Lactococcus diacetylactis, Lactococcus cremoris, Lactobacillus bulgaricus, Lactobacillus helveticus, Lactobacillus delbrueckii, Escherichia coli, and combinations thereof. In some embodiments, the gram positive bacteria is Escherichia coli. In some embodiments, the gram positive bacteria is a probiotic bacteria. In some embodiments, the gram positive bacteria is Escherichia coli Nissle. In some embodiments, the Escherichia coli Nissle comprises one or modifications in genes selected from the group consisting of: lpxL, lpxP, lpxM, crcA, eptA, lpxT, nlpl, recA, ompT, lon, lpxA, lpxB, lpxD, pagL, pagP, lpxE, MsbA, MsbB, gutQ, and KdsD. In some embodiments, the Escherichia coli Nissle comprises one or modifications in genes selected from the group consisting of: lpxL, lpxP, lpxM, crcA, eptA, lpxT, nlpl, recA, ompT, and lon.

In some embodiments, the MIV is capable of eliciting an immune response in a mammal without an adjuvant administration.

In some aspects of the disclosure, a multivalent immunogenic polypeptide (MIP) is provided, comprising at least five influenza virus immunogenic epitopes selected from the group consisting of Y₁, Y₂, Y₃, Y₄, Y₅, and Y₆.

In some embodiments, the at least five influenza virus immunogenic epitopes are fused in tandem. In some embodiments, the MIP comprises the amino acid sequence having a formula (M2e-1)-(M2e-2)-(M2e-3)-(M2e-4)-(M2e-5)-(M2e-6), wherein M2e-1 comprises human M2e peptide, wherein M2e-2 comprises swine M2e peptide, wherein M2e-3 comprises swan M2e peptide, wherein M2e-4 comprises chicken M2e peptide, wherein M2e-5 comprises chicken M2e peptide, and wherein M2e-6 comprises mallard M2e peptide. In some embodiments, the MIP comprises the amino acid sequence having a formula (M2e-1)-(M2e-2)-(M2e-3)-(M2e-4)-(M2e-5)-(M2e-6), wherein M2e-1 comprises the amino acid sequence at least about 90% identical to SEQ ID NO: 3, wherein M2e-2 comprises the amino acid sequence at least about 90% identical to SEQ ID NO: 4, wherein M2e-3 comprises the amino acid sequence at least about 90% identical to SEQ ID NO: 5, wherein M2e-4 comprises the amino acid sequence at least about 90% identical to SEQ ID NO: 6, wherein M2e-5 comprises the amino acid sequence at least about 90% identical to SEQ ID NO: 7, and wherein M2e-6 comprises the amino acid sequence at least about 90% identical to SEQ ID NO: 8.

In some aspects of the disclosure, a fusion protein comprising a transport protein and a MIP comprising at least five influenza virus immunogenic epitopes selected from the group consisting of Y₁, Y₂, Y₃, Y₄, Y₅, and Y₆ is provided.

In some embodiments, the MIP is fused to a C-terminus of a transport protein. In some embodiments, the amino acid sequence having a formula X₁-(M2e-1)-(M2e-2)-(M2e-3)-(M2e-4)-(M2e-5)-(M2e-6), wherein X₁ comprises ClyA, wherein M2e-1 comprises the amino acid sequence at least about 90% identical to SEQ ID NO: 3, wherein M2e-2 comprises the amino acid sequence at least about 90% identical to SEQ ID NO: 4, wherein M2e-3 comprises the amino acid sequence at least about 90% identical to SEQ ID NO: 5, wherein M2e-4 comprises the amino acid sequence at least about 90% identical to SEQ ID NO: 6, wherein M2e-5 comprises the amino acid sequence at least about 90% identical to SEQ ID NO: 7, and wherein M2e-6 comprises the amino acid sequence at least about 90% identical to SEQ ID NO: 8. In some embodiments, the MIP or the fusion protein of the disclosure is provided, wherein the MIP comprises the amino acid sequence at least about 80% identical to SEQ ID NO: 9.

In some aspects of the disclosure, a nucleic acid encoding a MIP or a fusion protein of the disclosure is provided.

In some aspects of the disclosure, a vector comprising a nucleic acid of the disclosure is provided. In some aspects of the disclosure, a bacterial cell comprising a vector of the disclosure is provided.

In some aspects of the disclosure, a bacterial cell expressing a MIP or a fusion protein of the disclosure is provided.

In some aspects of the disclosure, a method of manufacturing a multivalent influenza viral vaccine (MIV) is provided, comprising a) culturing Escherichia coli Nissle comprising one or more modifications and expressing the transport protein and a MIP of the disclosure; b) aerating the culture of step a) by providing about 5% to about 20% oxygen; and c) isolating the MIV from supernatant of the culture.

In some embodiments, a particle size of the MIV is between 180 nanometers (nm) and 200 nm as measured by dynamic light scattering (DLS). In some embodiments, at least about 50% of the bacterial-derived vesicles of the plurality of bacterial-derived vesicles are intact. In some embodiments, at least about 75% of the bacterial-derived vesicles of the plurality of bacterial-derived vesicles are intact. In some embodiments, at least about 90% of the bacterial-derived vesicles of the plurality of bacterial-derived vesicles are intact. In some embodiments, at least about 50% of the bacterial-derived vesicles of the plurality of bacterial-derived vesicles express the MIP. In some embodiments, at least about 75% of the bacterial-derived vesicles of the plurality of bacterial-derived vesicles express the MIP. In some embodiments, at least about 90% of the bacterial-derived vesicles of the plurality of bacterial-derived vesicles express the MIP.

In some embodiments, step a) comprises culturing the Escherichia coli Nissle in a bacterial medium. In some embodiments, step a) comprises culturing the Escherichia coli Nissle in a mammalian cell medium.

In some aspects of the disclosure, a pharmaceutical composition is provided, comprising a MIV for the disclosure and a pharmaceutically acceptable excipient.

In some aspects of the disclosure, a method of treating a subject at risk of contracting influenza is provided, comprising administering a suitable amount of the pharmaceutical composition of the disclosure to the subject. In some embodiments, the method does not comprise administering an adjuvant to the subject.

### BRIEF DESCRIPTION OF THE DRAWINGS

The disclosure is more completely understood with reference to the following drawings.

FIG. 1 shows models of exemplary fusions proteins.

FIG. 2 shows viral strains and resulting M2e peptides and peptides present in 6xM2e or 4xM2e. FIG. 2 discloses SEQ ID NOS 2-8, respectively, in order of appearance.

FIG. 3 shows viral strains and resulting M2e peptides. FIG. 3 discloses SEQ ID NOS 2-8, respectively, in order of appearance.

FIGS. 4A and 4B show models of protein disorder of in silico determination of disordered proteins for M2e1x, M2e2xM2e3xM2e4xM2e5x, and M2e6x. Using Phyre2, web portal for protein modeling, prediction and analysis.

FIG. 5A shows an exemplary SDS PAGE of a purified ClyA-6M2e protein. FIG. 5B shows an analytical size exclusion chromatography of exemplary OMVs.

FIG. 6 shows the design strategy for the E. coli Nissle strain.

FIGS. 7A and 7B VT-104 provided 100% protection in mice. FIG. 7A ClyA-M2 fusion protein with 4xM2e vaccine provides 100% protection. FIG. 7B Serum from M2e-immunized mice provides 100% protection against lethal challenge with influenza.

FIGS. 8A and 8B Antibody titers correlate with protection. FIG. 8A Mouse serum was analyzed for anti-M2e titers 8 weeks post initial vaccination. FIG. 8B Linear regression of anti-M2e IgG2a log transformed titers vs. minimum percent original weight of rOMV-immunized mice during PR8 challenge.

FIG. 9 shows the 4xM2e and 6xM2e Ferret H5N1 Efficacy Study Design. Challenge strain: Chicken egg derived; H5N1 (+) vaccine: Formalin/UV-inactivated, vero-cell derived.

FIG. 10 shows survival curves of ferrets that show survival of VT-105-immunized ferrets over VT-104-immunized ferrets when challenged with H5N1.

FIG. 11 shows graphs of antibody titers by ELISA in ferrets vaccinated with either VT-104 or VT-105.

### DETAILED DESCRIPTION

Described herein, in certain embodiments, are multivalent influenza virus vaccine (MIV), multivalent immunogenic polypeptide (MIP), and fusion proteins. Provided herein are further methods of making and using the MIV, MIP, and fusion proteins.

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

### Definitions

Unless defined otherwise, all technical and scientific terms used herein have the same meaning as is commonly understood by one of skill in the art to which the claimed subject matter belongs. Generally, nomenclatures utilized in connection with, and techniques of, immunology, oncology, cell and tissue culture, molecular biology, and protein and oligonucleotide or polynucleotide chemistry and hybridization described herein are those well-known and commonly used in the art. Units of measure not otherwise defined accord with The International System of Units (SI), NIST Special Publication 330, 2019 edition.

As used herein, all numerical values or numerical ranges comprise whole integers within or encompassing such ranges and fractions of the values or the integers within or encompassing ranges unless the context clearly indicates otherwise. Thus, for example, reference to a range of 90-100%, comprises 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000-fold comprises 1-, 2-, 3-, 4-, 5-, 6-, 7-, 8-, 9-, 10-, 11-, 12-, 13-, 14-, 15-, 16-, 17-, 18-, 19-, or 20-fold, etc., as well as 1.1-, 1.2-, 1.3-, 1.4-, or 1.5-fold, etc., 2.1-, 2.2-, 2.3-, 2.4-, or 2.5-fold, etc., and so forth.

The terminology used herein is for the purpose of describing particular embodiments only and is not intended to be limiting of any embodiment. As used herein, the singular forms “a,” “an,” and “the” are intended to comprise the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” comprises any and all combinations of one or more of the associated listed items.

Unless specifically stated or obvious from context, as used herein, the term “about” in reference to a number or range of numbers is understood to mean the stated number and numbers±10% thereof, or 10% below the lower listed limit and 10% above the higher listed limit for the values listed for a range.

As used herein, the term “immunogenic” refers to the ability of a substance to induce an immune response in a recipient. In some embodiments, an immune response is induced when an immune system of an organism or a certain type of immune cells is exposed to an immunogenic substance. The term “non-immunogenic” refers to a lack of or absence of an immune response above a detectable threshold to a substance. In some embodiments, no immune response is detected when an immune system of an organism or a certain type of immune cells is exposed to a non-immunogenic substance. In some embodiments, a non-immunogenic composition, vector, or nucleic acid of the disclosure is provided herein, which does not induce an immune response above a pre-determined threshold when measured by an immunogenicity assay. In some embodiments, a reduced-immunogenic composition, vector, or nucleic acid of the disclosure is provided herein, which induces a reduced immune response below a pre-determined threshold when measured by an immunogenicity assay. For example, when an immunogenicity assay is used to measure antibodies raised against inflammatory markers, a non-immunogenic or reduced-immunogenic composition as provided herein leads to production of antibodies or markers at a level lower than predetermined threshold. The predetermined threshold is, for instance, at most 1.5 times, 2 times, 3 times, 4 times, or 5 times the level of antibodies or markers raised by a control reference.

“Percent identity,” “% identity,” or “sequence identity” refer to the extent to which two sequences (nucleotide or amino acid) have the same residues at the same positions in an alignment. For example, “a nucleotide sequence is X % identical to SEQ ID NO: Y” refers to % identity of the nucleotide sequence to SEQ ID NO: Y and is elaborated as X % of residues in the nucleotide sequence are identical to the corresponding residues of sequence disclosed in SEQ ID NO: Y. A sequence said to be X % identical to a reference sequence may contain more nucleotide or amino acid residues than specified in the reference sequence but must contain a sequence corresponding to the reference sequence. In most cases, the sequence in question will contain a sequence that corresponds to all of the specified reference sequences. Generally, computer programs are employed for such calculations. Exemplary programs that compare and align pairs of sequences, comprise ALIGN, FASTA, gapped BLAST, BLASTP, BLASTN, or GCG.

The term “plasmid” refers to an extrachromosomal element that carries genes that can replicate independently of the chromosomes of the cell. The plasmid can be in the form of a circular double-stranded DNA molecule. Such elements can include autonomously replicating sequences, genome integrating sequences, phage or nucleotide sequences, as well as linear, circular or supercoiled, single- or double-stranded DNA or RNA of any origin. Exemplary plasmids include, but are not limited to, minicircles and doggybone plasmids.

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. Contemplated polynucleotides include a gene or fragment thereof. Exemplary polynucleotides include, but are not limited to, DNA, RNA, coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, cell-free polynucleotides including cell-free DNA (cfDNA) and cell-free RNA (cfRNA), nucleic acid probes, and primers. In a polynucleotide when referring to a T, a T means U (Uracil) in RNA and T (Thymine) in DNA. A polynucleotide can be exogenous or endogenous to a cell and/or exist in a cell-free environment. The term polynucleotide encompasses modified polynucleotides (e.g., altered backbone, sugar, or nucleobase). If present, modifications to the nucleotide structure are imparted before or after assembly of the polymer. Non-limiting examples of modifications include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol-containing nucleotides, biotin-linked nucleotides, fluorescent base analogs, CpG islands, methyl-7-guanosine, methylated nucleotides, inosine, thiouridine, pseudouridine, dihydrouridine, queuosine, and wyosine. The sequence of nucleotides may be interrupted by non-nucleotide components.

The phrase “pharmaceutically acceptable” refers to molecular entities and compositions that are physiologically tolerable to a mammal, particularly a human or animal patient.

As used herein, the term “vector” comprises a nucleic acid vector, e.g., a DNA vector, such as a plasmid, an RNA vector, or another suitable replicon (e.g., viral vector). A variety of vectors have been developed for the delivery of polynucleotides encoding exogenous polynucleotides or proteins into a prokaryotic or eukaryotic cell. Examples of such expression vectors are disclosed in, e.g., WO 1994/011026 as it pertains to vectors suitable for the expression of a nucleic acid molecule of interest. Expression vectors suitable for use with the compositions and methods described herein contain a polynucleotide sequence as well as, e.g., additional sequence elements used for the expression of heterologous nucleic acid materials (e.g., a nucleic acid molecule) in a cell. Certain vectors that are used for the expression of the nucleic acid molecules described herein comprise plasmids that contain regulatory sequences, such as promoter and enhancer regions, which direct gene transcription. In some embodiments, the compact bidirectional promoters do not contain an enhancer. Other useful vectors for expression of nucleic acid molecule agents disclosed herein contain polynucleotide sequences that enhance the rate of translation of these polynucleotides or improve the stability or nuclear export of the RNA that results from gene transcription. These sequence elements comprise, e.g., 5′ and 3′ untranslated regions, an internal ribosomal entry site (IRES), and polyadenylation signal (polyA) in order to direct efficient transcription of the gene carried on the expression vector. In some embodiments, the expression vectors suitable for use with the compositions and methods described herein contain a backbone polynucleotide encoding a marker for selection of cells that contain such a vector. Examples of a suitable marker are genes that encode resistance to antibiotics, such as ampicillin, chloramphenicol, neomycin, zeocin, kanamycin, nourseothricin, aminoglycoside, a beta-lactam, a glycopeptide, a macrolide, a polypeptide, a tetracycline, spectinomycin, streptomycin, carbenicillin, bleomycin, erythromycin, polymyxin B, chloramphenicol, or a derivative thereof.

The term “immunization” refers to a process that increases a mammalian subject's reaction to an antigen and therefore improves its ability to resist or overcome infection and/or resist disease.

The term “vaccination” as used herein refers to the introduction of a vaccine into a body of a subject, preferably, a mammalian subject such as a human.

### Multivalent Influenza Virus Vaccine Compositions

Described herein, in certain embodiments, are multivalent influenza virus vaccines (MIV) and multivalent immunogenic polypeptides (MIP).

In some embodiments, the MIV comprises: A-B-C wherein i) A is a transport protein or a fragment thereof comprising at least domains X₁ and X₂, wherein a) X₁ is a transmembrane domain polypeptide, and b) X₂ is a polypeptide capable of being operably linked to B; ii) B is a multivalent immunogenic polypeptide (MIP) comprising at least five influenza virus immunogenic epitopes selected from the group consisting of Y₁, Y₂, Y₃, Y₄, Y₅, and Y₆, iii) C is a membrane vesicle derived from a genetically modified gram positive bacteria; wherein A and B are operably linked, resulting in a multivalent immunogenic transmembrane polypeptide; wherein A-B is linked to C via the transmembrane domain X₁, thereby forming the MIV; and wherein the MIV is capable of eliciting an immune response to at least two influenza strains when administered to a mammal.

In some embodiments, the multivalent influenza virus vaccine (MIV), comprises: a) a transport protein or a fragment thereof, wherein said transport protein comprises a transmembrane domain; b) a multivalent immunogenic polypeptide (MIP) comprising at least five covalently linked influenza virus immunogenic epitopes; c) a membrane vesicle derived from a genetically modified gram positive bacteria; wherein the transport protein of a) is operably linked to the MIP of b), resulting in a multivalent immunogenic transmembrane polypeptide; wherein the multivalent immunogenic transmembrane polypeptide is linked to the vesicle forming the MIV; and wherein the MIV is capable of eliciting an immune response to at least 2 influenza virus strains when administered to a mammal.

In some embodiments, the MIV is capable of eliciting an immune response in a mammal without an adjuvant administration.

### Immunogenic Polypeptides

Described herein, in some embodiments, are MIVs comprising immunogenic polypeptides or epitopes. In some embodiments, the MIV comprises at least five or more immunogenic epitopes. In some embodiments, immunogenic epitopes are influenza virus immunogenic epitopes. In some embodiments, the influenza virus is influenza A. In some embodiments, the influenza A is human, swine, or avian. In some embodiments, the avian is a chicken, a whooper a swan, a quail, or a mallard. In some embodiments, influenza A is influenza subtype is H1, H2, H3, H5, H6, H7, or H9. In some embodiments, the influenza virus is selected from the group consisting of H1N1, H1N2, H2N1, H3N2, H5N1, H5N2, H9N2, H7N9, H7N7, H7N3, H6N6, H6N2, and H6N1.

In some embodiments, the at least five influenza virus immunogenic epitopes are different. In some embodiments, the at least five influenza virus immunogenic epitopes are the same. In some embodiments, at least one of the at least five influenza virus immunogenic epitopes comprises a consensus sequence or a non-naturally occurring sequence.

In some embodiments, the at least five influenza virus immunogenic epitopes are fused in tandem. In some embodiments, the operable linkage between A and B comprises a covalent linkage. In some embodiments, the at least five influenza virus immunogenic epitopes are fused to a N- or C-terminus of the transport protein. In some embodiments, the at least five influenza virus immunogenic epitopes are presented on outside of the vesicle.

In some embodiments, the MIP comprises an influenza A matrix protein 2 extracellular (M2e) peptide or fragment thereof. In some embodiments, the MIP comprises five M2e peptides. In some embodiments, the MIP comprises six M2e peptides. In some embodiments, the M2e peptide comprises the amino acid sequence at least about 90% identical to any one of SEQ ID NOs: 2-8. In some embodiments, Y1, Y2, Y3, Y4, Y5, and Y6 are selected from the group consisting of a human M2e peptide, a swine M2e peptide, a swan M2e peptide, a chicken M2e peptide, and a mallard M2e peptide.

In some embodiments, the multivalent immunogenic transmembrane polypeptide comprises the amino acid sequence having a formula ClyA-(M2e)5. In some embodiments, the multivalent immunogenic transmembrane polypeptide comprises the amino acid sequence having a formula ClyA-(M2e)6. In some embodiments, the multivalent immunogenic transmembrane polypeptide comprises the amino acid sequence having a formula ClyA-(M2e-1)-(M2e-2)-(M2e-3)-(M2e-4)-(M2e-5)-(M2e-6), wherein M2e-1 comprises human M2e peptide, wherein M2e-2 comprises swine M2e peptide, wherein M2e-3 comprises swan M2e peptide, wherein M2e-4 comprises chicken M2e peptide, wherein M2e-5 comprises chicken M2e peptide, and wherein M2e-6 comprises mallard M2e peptide. In some embodiments, the multivalent immunogenic transmembrane polypeptide comprises the amino acid sequence having a formula ClyA-(M2e-1)-(M2e-2)-(M2e-3)-(M2e-4)-(M2e-5)-(M2e-6), wherein M2e-1 comprises the amino acid sequence at least about 90% identical to SEQ ID NO: 3, wherein M2e-2 comprises the amino acid sequence at least about 90% identical to SEQ ID NO: 4, wherein M2e-3 comprises the amino acid sequence at least about 90% identical to SEQ ID NO: 5, wherein M2e-4 comprises the amino acid sequence at least about 90% identical to SEQ ID NO: 6, wherein M2e-5 comprises the amino acid sequence at least about 90% identical to SEQ ID NO: 7, and wherein M2e-6 comprises the amino acid sequence at least about 90% identical to SEQ ID NO: 8.

In some embodiments, the multivalent immunogenic transmembrane polypeptide comprises the amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 9. In some embodiments, the multivalent immunogenic transmembrane polypeptide comprises the amino acid sequence at least 70% identical to SEQ ID NO: 9. In some embodiments, the multivalent immunogenic transmembrane polypeptide comprises the amino acid sequence at least 80% identical to SEQ ID NO: 9. In some embodiments, the multivalent immunogenic transmembrane polypeptide comprises the amino acid sequence at least 85% identical to SEQ ID NO: 9. In some embodiments, the multivalent immunogenic transmembrane polypeptide comprises the amino acid sequence at least 90% identical to SEQ ID NO: 9. In some embodiments, the multivalent immunogenic transmembrane polypeptide comprises the amino acid sequence at least 70% identical to SEQ ID NO: 9. In some embodiments, the multivalent immunogenic transmembrane polypeptide comprises the amino acid sequence at least 91% identical to SEQ ID NO: 9. In some embodiments, the multivalent immunogenic transmembrane polypeptide comprises the amino acid sequence at least 92% identical to SEQ ID NO: 9. In some embodiments, the multivalent immunogenic transmembrane polypeptide comprises the amino acid sequence at least 93% identical to SEQ ID NO: 9. In some embodiments, the multivalent immunogenic transmembrane polypeptide comprises the amino acid sequence at least 94% identical to SEQ ID NO: 9. In some embodiments, the multivalent immunogenic transmembrane polypeptide comprises the amino acid sequence at least 95% identical to SEQ ID NO: 9. In some embodiments, the multivalent immunogenic transmembrane polypeptide comprises the amino acid sequence at least 96% identical to SEQ ID NO: 9. In some embodiments, the multivalent immunogenic transmembrane polypeptide comprises the amino acid sequence at least 97% identical to SEQ ID NO: 9. In some embodiments, the multivalent immunogenic transmembrane polypeptide comprises the amino acid sequence at least 98% identical to SEQ ID NO: 9. In some embodiments, the multivalent immunogenic transmembrane polypeptide comprises the amino acid sequence at least 99% identical to SEQ ID NO: 9. In some embodiments, the multivalent immunogenic transmembrane polypeptide comprises the amino acid sequence identical to SEQ ID NO: 9.

### Transport Protein

Described herein, in some embodiments, are multivalent influenza virus vaccines (MIV), said MIV comprising a transport protein (A) or a fragment thereof. In some embodiments, the transport protein is an adhesin, immunomodulatory compound, protease, or toxin, or a fragment thereof.

In some embodiments, the transport protein is ClyA. In some embodiments, the ClyA comprises the amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 1. In some embodiments, the ClyA comprises the amino acid sequence at least 70% identical to SEQ ID NO: 1. In some embodiments, the ClyA comprises the amino acid sequence at least 80% identical to SEQ ID NO: 1. In some embodiments, the ClyA comprises the amino acid sequence at least 85% identical to SEQ ID NO: 1. In some embodiments, the ClyA comprises the amino acid sequence at least 90% identical to SEQ ID NO: 1. In some embodiments, the ClyA comprises the amino acid sequence at least 70% identical to SEQ ID NO: 1. In some embodiments, the ClyA comprises the amino acid sequence at least 91% identical to SEQ ID NO: 1. In some embodiments, the ClyA comprises the amino acid sequence at least 92% identical to SEQ ID NO: 1. In some embodiments, the ClyA comprises the amino acid sequence at least 93% identical to SEQ ID NO: 1. In some embodiments, the ClyA comprises the amino acid sequence at least 94% identical to SEQ ID NO: 1. In some embodiments, the ClyA comprises the amino acid sequence at least 95% identical to SEQ ID NO: 1. In some embodiments, the ClyA comprises the amino acid sequence at least 96% identical to SEQ ID NO: 1. In some embodiments, the ClyA comprises the amino acid sequence at least 97% identical to SEQ ID NO: 1. In some embodiments, the ClyA comprises the amino acid sequence at least 98% identical to SEQ ID NO: 1. In some embodiments, the ClyA comprises the amino acid sequence at least 99% identical to SEQ ID NO: 1. In some embodiments, the ClyA comprises the amino acid sequence identical to SEQ ID NO: 1.

### Multivalent Immunogenic Polypeptide (MIP)

Described herein, in some embodiments, are multivalent immunogenic polypeptides (MIP) comprising immunogenic polypeptides or epitopes.

In some embodiments, the MIP comprises at least five or more immunogenic epitopes. In some embodiments, immunogenic epitopes are influenza virus immunogenic epitopes. In some embodiments, the influenza virus is influenza A. In some embodiments, the influenza A is human, swine, or avian. In some embodiments, the avian is a chicken, a whooper a swan, a quail, or a mallard. In some embodiments, influenza A is influenza subtype is H1, H2, H3, H5, H6, H7, or H9. In some embodiments, the influenza virus is selected from the group consisting of H1N1, H1N2, H2N1, H3N2, H5N1, H5N2, H9N2, H7N9, H7N7, H7N3, H6N6, H6N2, and H6N1.

In some embodiments, the multivalent immunogenic polypeptide (MIP) comprises at least five influenza virus immunogenic epitopes selected from the group consisting of Y1, Y2, Y3, Y4, Y5, and Y6.

In some embodiments, the at least five influenza virus immunogenic epitopes are different. In some embodiments, the at least five influenza virus immunogenic epitopes are the same. In some embodiments, at least one of the at least five influenza virus immunogenic epitopes comprises a consensus sequence or a non-naturally occurring sequence.

In some embodiments, the at least five influenza virus immunogenic epitopes are fused in tandem.

In some embodiments, the MIP comprises an influenza A matrix protein 2 extracellular (M2e) peptide or fragment thereof. In some embodiments, the MIP comprises five M2e peptides. In some embodiments, the MIP comprises six M2e peptides. In some embodiments, the M2e peptide comprises the amino acid sequence at least about 70% identical (e.g., at least about 70%, 75%, 80%, 85%, 90%, 95%, 96%, 97%, 98%, 99%) to any one of SEQ ID NOs: 2-8. In some embodiments, Y1, Y2, Y3, Y4, Y5, and Y6 are selected from the group consisting of a human M2e peptide, a swine M2e peptide, a swan M2e peptide, a chicken M2e peptide, and a mallard M2e peptide. In some embodiments, the MIP comprises the amino acid sequence having a formula (M2e-1)-(M2e-2)-(M2e-3)-(M2e-4)-(M2e-5)-(M2e-6), wherein M2e-1 comprises human M2e peptide, wherein M2e-2 comprises swine M2e peptide, wherein M2e-3 comprises swan M2e peptide, wherein M2e-4 comprises chicken M2e peptide, wherein M2e-5 comprises chicken M2e peptide, and wherein M2e-6 comprises mallard M2e peptide. In some embodiments, the MIP comprises the amino acid sequence having a formula (M2e-1)-(M2e-2)-(M2e-3)-(M2e-4)-(M2e-5)-(M2e-6), wherein M2e-1 comprises the amino acid sequence at least about 70% identical to SEQ ID NO: 3, wherein M2e-2 comprises the amino acid sequence at least about 70% identical to SEQ ID NO: 4, wherein M2e-3 comprises the amino acid sequence at least about 70% identical to SEQ ID NO: 5, wherein M2e-4 comprises the amino acid sequence at least about 70% identical to SEQ ID NO: 6, wherein M2e-5 comprises the amino acid sequence at least about 70% identical to SEQ ID NO: 7, and wherein M2e-6 comprises the amino acid sequence at least about 70% identical to SEQ ID NO: 8.

In some embodiments, the disclosure provides for a fusion protein comprising a transport protein and a MIP comprising at least five influenza virus immunogenic epitopes selected from the group consisting of Y1, Y2, Y3, Y4, Y5, and Y6. In some embodiments, the MIP is fused to a C-terminus of a transport protein. In some embodiments, the fusion protein comprises the amino acid sequence having a formula X1-(M2e-1)-(M2e-2)-(M2e-3)-(M2e-4)-(M2e-5)-(M2e-6), wherein X1 comprises ClyA, wherein M2e-1 comprises the amino acid sequence at least about 70% identical to SEQ ID NO: 3, wherein M2e-2 comprises the amino acid sequence at least about 70% identical to SEQ ID NO: 4, wherein M2e-3 comprises the amino acid sequence at least about 70% identical to SEQ ID NO: 5, wherein M2e-4 comprises the amino acid sequence at least about 70% identical to SEQ ID NO: 6, wherein M2e-5 comprises the amino acid sequence at least about 70% identical to SEQ ID NO: 7, and wherein M2e-6 comprises the amino acid sequence at least about 70% identical to SEQ ID NO: 8. In some embodiments, the MIP or the fusion protein comprises the amino acid sequence at least about 80% identical to SEQ ID NO: 9.

In some embodiments, the MIP comprises an influenza A matrix protein 2 extracellular (M2e) peptide or fragment thereof. In some embodiments, the MIP comprises five M2e peptides. In some embodiments, the MIP comprises six M2e peptides. In some embodiments, the M2e peptide comprises the amino acid sequence at least about 80% identical to any one of SEQ ID NOs: 2-8. In some embodiments, Y1, Y2, Y3, Y4, Y5, and Y6 are selected from the group consisting of a human M2e peptide, a swine M2e peptide, a swan M2e peptide, a chicken M2e peptide, and a mallard M2e peptide. In some embodiments, the MIP comprises the amino acid sequence having a formula (M2e-1)-(M2e-2)-(M2e-3)-(M2e-4)-(M2e-5)-(M2e-6), wherein M2e-1 comprises human M2e peptide, wherein M2e-2 comprises swine M2e peptide, wherein M2e-3 comprises swan M2e peptide, wherein M2e-4 comprises chicken M2e peptide, wherein M2e-5 comprises chicken M2e peptide, and wherein M2e-6 comprises mallard M2e peptide. In some embodiments, the MIP comprises the amino acid sequence having a formula (M2e-1)-(M2e-2)-(M2e-3)-(M2e-4)-(M2e-5)-(M2e-6), wherein M2e-1 comprises the amino acid sequence at least about 80% identical to SEQ ID NO: 3, wherein M2e-2 comprises the amino acid sequence at least about 80% identical to SEQ ID NO: 4, wherein M2e-3 comprises the amino acid sequence at least about 80% identical to SEQ ID NO: 5, wherein M2e-4 comprises the amino acid sequence at least about 80% identical to SEQ ID NO: 6, wherein M2e-5 comprises the amino acid sequence at least about 80% identical to SEQ ID NO: 7, and wherein M2e-6 comprises the amino acid sequence at least about 80% identical to SEQ ID NO: 8.

In some embodiments, the disclosure provides for a fusion protein comprising a transport protein and a MIP comprising at least five influenza virus immunogenic epitopes selected from the group consisting of Y1, Y2, Y3, Y4, Y5, and Y6. In some embodiments, the MIP is fused to a C-terminus of a transport protein. In some embodiments, the fusion protein comprises the amino acid sequence having a formula X1-(M2e-1)-(M2e-2)-(M2e-3)-(M2e-4)-(M2e-5)-(M2e-6), wherein X1 comprises ClyA, wherein M2e-1 comprises the amino acid sequence at least about 80% identical to SEQ ID NO: 3, wherein M2e-2 comprises the amino acid sequence at least about 80% identical to SEQ ID NO: 4, wherein M2e-3 comprises the amino acid sequence at least about 80% identical to SEQ ID NO: 5, wherein M2e-4 comprises the amino acid sequence at least about 80% identical to SEQ ID NO: 6, wherein M2e-5 comprises the amino acid sequence at least about 80% identical to SEQ ID NO: 7, and wherein M2e-6 comprises the amino acid sequence at least about 80% identical to SEQ ID NO: 8. In some embodiments, the MIP or the fusion protein comprises the amino acid sequence at least about 80% identical to SEQ ID NO: 9.

In some embodiments, the MIP comprises an influenza A matrix protein 2 extracellular (M2e) peptide or fragment thereof. In some embodiments, the MIP comprises five M2e peptides. In some embodiments, the MIP comprises six M2e peptides. In some embodiments, the M2e peptide comprises the amino acid sequence at least about 90% identical to any one of SEQ ID NOs: 2-8. In some embodiments, Y1, Y2, Y3, Y4, Y5, and Y6 are selected from the group consisting of a human M2e peptide, a swine M2e peptide, a swan M2e peptide, a chicken M2e peptide, and a mallard M2e peptide. In some embodiments, the MIP comprises the amino acid sequence having a formula (M2e-1)-(M2e-2)-(M2e-3)-(M2e-4)-(M2e-5)-(M2e-6), wherein M2e-1 comprises human M2e peptide, wherein M2e-2 comprises swine M2e peptide, wherein M2e-3 comprises swan M2e peptide, wherein M2e-4 comprises chicken M2e peptide, wherein M2e-5 comprises chicken M2e peptide, and wherein M2e-6 comprises mallard M2e peptide. In some embodiments, the MIP comprises the amino acid sequence having a formula (M2e-1)-(M2e-2)-(M2e-3)-(M2e-4)-(M2e-5)-(M2e-6), wherein M2e-1 comprises the amino acid sequence at least about 90% identical to SEQ ID NO: 3, wherein M2e-2 comprises the amino acid sequence at least about 90% identical to SEQ ID NO: 4, wherein M2e-3 comprises the amino acid sequence at least about 90% identical to SEQ ID NO: 5, wherein M2e-4 comprises the amino acid sequence at least about 90% identical to SEQ ID NO: 6, wherein M2e-5 comprises the amino acid sequence at least about 90% identical to SEQ ID NO: 7, and wherein M2e-6 comprises the amino acid sequence at least about 90% identical to SEQ ID NO: 8.

In some embodiments, the disclosure provides for a fusion protein comprising a transport protein and a MIP comprising at least five influenza virus immunogenic epitopes selected from the group consisting of Y1, Y2, Y3, Y4, Y5, and Y6. In some embodiments, the MIP is fused to a C-terminus of a transport protein. In some embodiments, the fusion protein comprises the amino acid sequence having a formula X1-(M2e-1)-(M2e-2)-(M2e-3)-(M2e-4)-(M2e-5)-(M2e-6), wherein X1 comprises ClyA, wherein M2e-1 comprises the amino acid sequence at least about 90% identical to SEQ ID NO: 3, wherein M2e-2 comprises the amino acid sequence at least about 90% identical to SEQ ID NO: 4, wherein M2e-3 comprises the amino acid sequence at least about 90% identical to SEQ ID NO: 5, wherein M2e-4 comprises the amino acid sequence at least about 90% identical to SEQ ID NO: 6, wherein M2e-5 comprises the amino acid sequence at least about 90% identical to SEQ ID NO: 7, and wherein M2e-6 comprises the amino acid sequence at least about 90% identical to SEQ ID NO: 8. In some embodiments, the MIP or the fusion protein comprises the amino acid sequence at least about 80% identical to SEQ ID NO: 9.

In some embodiments, the MIV and/or the MIP are designed in silico.

Advantages of the in silico design of a multi-epitope fusion protein may comprise, but are not limited to, higher genetic diversity of influenza strains represented, improved magnitude of the immune response, improved quality of the antibody response, improved cross-strain protection, higher potency, and reduced toxicity associated with LPS.

In some embodiments, the in silico designed MIPs are probed in silico for the degree of disorder after protein folding.

Disordered antigens may comprise various advantages including, but not limited to, improved efficiency of antibody binding to disordered epitopes, more agility of disordered epitopes to bind an antibody, more energetically favored, larger interface to contact the antibody, more favorable enthalpic (i.e., electrostatic interactions), enrichment of polar contact residues, more solvent exposed than ordered/tertiary structured epitopes, and increased hydrogen bonds and salt bridges per antigen residue.

### Membrane Vesicles

Described herein, in some embodiments, are multivalent influenza virus vaccines (MIV), comprising membrane vesicles. In some embodiments, the membrane vesicles are derived from a gram positive bacteria. In some embodiments, the gram positive bacteria is selected from the group consisting of: Bifidobacterium longum, Bifidobacterium lactis, Bifidobacterium animalis, Bifidobacterium breve, Bifidobacterium infantis, Bifidobacterium adolescentis, Lactobacillus acidophilus, Lactobacillus casei, Lactobacillus paracasei, Lactobacillus salivarius, Lactobacillus reuteri, Lactobacillus rhamnosus, Lactobacillus johnsonii, Lactobacillus plantarum, Lactobacillus fermentum, Lactococcus lactis, Streptococcus thermophilus, Lactococcus lactis, Lactococcus diacetylactis, Lactococcus cremoris, Lactobacillus bulgaricus, Lactobacillus helveticus, Lactobacillus delbrueckii, Escherichia coli, and combinations thereof. In some embodiments, the gram positive bacteria is Escherichia coli. In some embodiments, the gram positive bacteria is a probiotic bacteria. In some embodiments, the gram positive bacteria is Escherichia coli Nissle. In some embodiments, the Escherichia coli Nissle comprises one or modifications in genes selected from the group consisting of: lpxL, lpxP, lpxM, crcA, eptA, lpxT, nlpl, recA, ompT, lon, lpxA, lpxB, lpxD, pagL, pagP, lpxE, MsbA, MsbB, gutQ, and KdsD. In some embodiments, the Escherichia coli Nissle comprises one or modifications in genes selected from the group consisting of: lpxL, lpxP, lpxM, crcA, eptA, lpxT, nlpl, recA, ompT, and lon.

### Linkers Sequences

Described herein, in some embodiments, are multivalent influenza virus vaccines (MIV), wherein the components of the MIV are linked using one or more linkers. In some embodiments, the transport protein and the multivalent immunogenic polypeptide (MIP) are linked using one or more linkers.

In some embodiments, the one or more linkers comprises at least 5 to about 50 amino acids. In some embodiments, one or both of the first linker and the second linker comprises about 5 to about 50 amino acids, about 5 to about 45 amino acids, about 5 to about 40 amino acids, about 5 to about 35 amino acids, about 5 to about 30 amino acids, about 5 to about 25 amino acids, about 5 to about 20 amino acids, about 5 to about 15 amino acids, about 5 to about 10 amino acids, about 10 to about 50 amino acids, about 15 to about 50 amino acids, about 20 to about 50 amino acids, about 25 to about 50 amino acids, about 30 to about 50 amino acids, about 35 to about 50 amino acids, about 40 to about 50 amino acids, or about 45 to about 50 amino acids.

In some embodiments, the one or more linkers comprises a sequence selected from the group consisting of (GS)n (SEQ ID NO: 12), (G2S)n (SEQ ID NO: 13), (G3S)n (SEQ ID NO: 14), (G4S)n (SEQ ID NO: 15), and (G)n (SEQ ID NO: 16), and wherein n is an integer from 2 to 20. In some embodiments, n is an integer from 2 to 18, from 2 to 16, from 2 to 14, from 2 to 12, from 2 to 10, from 2 to 8, from 2 to 6, from 2 to 4, from 4 to 20, from 6 to 20, from 8 to 20, from 10 to 20, from 12 to 20, from 14 to 20, from 16 to 20, or from 18 to 20.

In some embodiments, the one or more linkers comprises a sequence selected from the group consisting of (GGSGGD)n (SEQ ID NO: 17) or (GGSGGE)n (SEQ ID NO: 18), and wherein n is an integer from 2 to 6.

In some embodiments, the one or more linkers comprises a sequence selected from the group consisting of (GGGSGSGGGGS)n (SEQ ID NO: 20) and (GGGGGPGGGGP)n (SEQ ID NO: 21), and wherein n is an integer from 1 to 3.

In some embodiments, the one or more linkers comprises a sequence selected from the group consisting of (GGGSGGG)n (SEQ ID NO: 19), (GGGSGSGGGGS)n (SEQ ID NO: 20) and (GGGGGPGGGGP)n (SEQ ID NO: 21), and wherein n is an integer from 1 to 3.

In some embodiments, the one or more linkers comprises a sequence selected from the group consisting of (GX)n, (GGX)n, (GGGX)n, (GGGGX)n, and (GzX)n, wherein z is between 1 and 20, and wherein n is at least 8. In some embodiments, z is between 2 and 18, 2 and 16, 2 and 14, 2 and 12, 2 and 10, 2 and 8, 2 and 6, 2 and 4, 4 and 20, 6 and 20, 8 and 20, 10 and 20, 12 and 20, 14 and 20, 16 and 20, or 18 and 20. In some embodiments, X is serine, aspartic acid, glutamic acid, threonine, or proline.

In some embodiments, the one or more linkers is GGGSGGG (SEQ ID NO: 22).

### Methods of Use and Manufacturing

Described herein, in some embodiments, are methods of use and manufacturing of multivalent influenza virus vaccines (MIV).

In some embodiments, the MIP or the fusion protein is encoded in a nucleic acid. In some embodiments, the disclosure provides for a vector comprising the nucleic acid. In some embodiments, the disclosure provides for a bacteria cell comprising the nucleic acid or the vector. In some embodiments, the bacterial cell expresses the MIP or the fusion protein of the disclosure.

In some embodiments, a method of manufacturing a multivalent influenza viral vaccine (MIV), is provided, comprising a) culturing Escherichia coli Nissle comprising one or more modifications and expressing the transport protein and the MIP of the disclosure; b) aerating the culture of step a) by providing about 5% to about 20% oxygen; and c)isolating the MIV from supernatant of the culture.

In some embodiments, the particle size of the MIV produced by the method is between 180 nanometers (nm) and 200 nm as measured by dynamic light scattering (DLS). In some embodiments, the particle size of the MIV produced by the method is at least or about 170, 175, 180, 185, 190, 195, 200, 205, 210, or more than 210 nm. In some embodiments, the particle size of the MIV produced by the method is between 170 nanometers (nm) and 210 nm, between 170 nm and 200 nm, between 180 nm and 190 nm, between 180 nm and 210 nm, between 180 nm and 200 nm, between 180 nm and 190 nm, between 190 nm and 210 nm, and between 190 nm and 200 nm.

In some embodiments, at least about 50% of the bacterial-derived vesicles of the plurality of bacterial-derived vesicles are intact. In some embodiments, at least about 55% of the bacterial-derived vesicles of the plurality of bacterial-derived vesicles are intact. In some embodiments, at least about 60% of the bacterial-derived vesicles of the plurality of bacterial-derived vesicles are intact. In some embodiments, at least about 65% of the bacterial-derived vesicles of the plurality of bacterial-derived vesicles are intact. In some embodiments, at least about 75% of the bacterial-derived vesicles of the plurality of bacterial-derived vesicles are intact. In some embodiments, at least about 80% of the bacterial-derived vesicles of the plurality of bacterial-derived vesicles are intact. In some embodiments, at least about 85% of the bacterial-derived vesicles of the plurality of bacterial-derived vesicles are intact. In some embodiments, at least about 90% of the bacterial-derived vesicles of the plurality of bacterial-derived vesicles are intact. In some embodiments, at least about 95% of the bacterial-derived vesicles of the plurality of bacterial-derived vesicles are intact. In some embodiments, at least about 97% of the bacterial-derived vesicles of the plurality of bacterial-derived vesicles are intact. In some embodiments, at least about 99% of the bacterial-derived vesicles of the plurality of bacterial-derived vesicles are intact.

In some embodiments, at least about 50% of the bacterial-derived vesicles of the plurality of bacterial-derived vesicles express the MIP. In some embodiments, at least about 55% of the bacterial-derived vesicles of the plurality of bacterial-derived vesicles express the MIP. In some embodiments, at least about 60% of the bacterial-derived vesicles of the plurality of bacterial-derived vesicles express the MIP. In some embodiments, at least about 65% of the bacterial-derived vesicles of the plurality of bacterial-derived vesicles express the MIP. In some embodiments, at least about 70% of the bacterial-derived vesicles of the plurality of bacterial-derived vesicles express the MIP. In some embodiments, at least about 75% of the bacterial-derived vesicles of the plurality of bacterial-derived vesicles express the MIP. In some embodiments, at least about 80% of the bacterial-derived vesicles of the plurality of bacterial-derived vesicles express the MIP. In some embodiments, at least about 85% of the bacterial-derived vesicles of the plurality of bacterial-derived vesicles express the MIP. In some embodiments, at least about 90% of the bacterial-derived vesicles of the plurality of bacterial-derived vesicles express the MIP. In some embodiments, at least about 95% of the bacterial-derived vesicles of the plurality of bacterial-derived vesicles express the MIP. In some embodiments, at least about 97% of the bacterial-derived vesicles of the plurality of bacterial-derived vesicles express the MIP. In some embodiments, at least about 99% of the bacterial-derived vesicles of the plurality of bacterial-derived vesicles express the MIP.

In some embodiments, step a) comprises culturing the Escherichia coli Nissle in a bacterial medium. In some embodiments, wherein step a) comprises culturing the Escherichia coli Nissle in a mammalian cell medium.

### Pharmaceutical Compositions

Described herein, in some embodiments, are pharmaceutical compositions comprising the multivalent influenza viral vaccine. In some embodiments, a pharmaceutical composition comprising a MIV of the disclosure and a pharmaceutically acceptable excipient are provided.

A composition or a medicament of the disclosure is in a form suitable for administration to an individual in need thereof.

According to a particular embodiment, the pharmaceutically acceptable excipient compositions of the disclosure are suitably selected from the group consisting of an injectable excipient liquid such as sterile water for injection; and an aqueous solution such as saline.

Acceptable excipients are physiologically acceptable to the administered subject and retain the therapeutic properties of the compounds with/in which it is administered. Acceptable excipients and their formulations are and generally described in, for example, Remington's Pharmaceutical Sciences, supra. One exemplary excipient is physiological saline. The phrase “pharmaceutically acceptable excipient” as used herein means a pharmaceutically acceptable material, composition or vehicle, such as a liquid or solid filler, diluent, excipient, solvent or encapsulating material, involved in carrying or transporting the subject compounds from the administration site of one organ, or portion of the body, to another organ, or portion of the body, or in an in vitro assay system. Each excipient is acceptable in the sense of being compatible with the other ingredients of the formulation and not injurious to a subject to whom it is administered. Nor should an acceptable excipient alter the specific activity of the subject compounds.

In another embodiment, pharmaceutical compositions disclosed herein further comprise an acceptable additive to improve the stability of the compounds in composition and/or to control the release rate of the compositions. Acceptable additives do not alter the specific activity of the subject compounds. Exemplary acceptable additives include, but are not limited to, a sugar such as mannitol, sorbitol, glucose, xylitol, trehalose, sorbose, sucrose, galactose, dextran, dextrose, fructose, lactose, and mixtures thereof. Acceptable additives are combined with acceptable carriers and/or excipients such as dextrose in some embodiments. Alternatively, exemplary acceptable additives include, but are not limited to, a surfactant such as polysorbate 20 or polysorbate 80 to increase stability of the peptide and decrease gelling of the solution. In some embodiments, the surfactant is added to the composition in an amount of 0.01% to 5% of the solution. Addition of such acceptable additives increases the stability and half-life of the composition in storage.

Suspensions, lyophilized, and crystal forms of nucleic acids or compositions herein are also contemplated herein; methods to make suspensions, lyophilizations, and crystal forms are known to one of skill in the art.

In some embodiments, pharmaceutical compositions disclosed herein are sterile. In some embodiments, pharmaceutical compositions disclosed herein are sterilized by conventional, well known sterilization techniques. For example, sterilization is readily accomplished by filtration through sterile filtration membranes. In some embodiments, the resulting solution is packaged for use or filtered under aseptic conditions and lyophilized, the lyophilized preparation being combined with a sterile solution prior to administration.

Freeze-drying is employed to stabilize polypeptides for long-term storage, such as when a polypeptide is relatively unstable in liquid compositions, in some embodiments.

In some embodiments, some excipients such as, for example, polyols (including mannitol, sorbitol, and glycerol); sugars (including glucose and sucrose); and amino acids (including alanine, glycine, and glutamic acid), act as stabilizers for freeze-dried products. Polyols and sugars are also used to protect polypeptides from freezing and drying-induced damage and to enhance the stability during storage in the dried state in some embodiments. Sugars are, in some embodiments, effective in both the freeze-drying process and during storage. Other classes of molecules, including mono- and disaccharides and polymers such as PVP, have also been reported as stabilizers of lyophilized products.

For injection, in some embodiments, pharmaceutical compositions disclosed herein are in a powder suitable for reconstitution with an appropriate solution as described above. Examples of these include, but are not limited to, freeze dried, rotary dried or spray dried powders, amorphous powders, granules, precipitates, or particulates. For injection, the compositions optionally contain stabilizers, pH modifiers, surfactants, bioavailability modifiers and combinations of these.

Sustained-release preparations are prepared in some embodiments. Suitable examples of sustained-release preparations include semipermeable matrices of solid hydrophobic polymers containing pharmaceutical compositions herein, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (see, e.g., U.S. Pat. No. 3,773,919), copolymers of L-glutamic acid and y ethyl-L-glutamate, non-degradable ethylene-vinyl acetate, degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

In some embodiments, pharmaceutical compositions disclosed herein are designed to be short-acting, fast-releasing, long-acting, or sustained-releasing as described herein. In one embodiment, pharmaceutical compositions disclosed herein are formulated for controlled release or for slow release.

In some embodiments, the pharmaceutical compositions are comprised in a container, pack, or dispenser together with instructions for administration.

In some embodiments, a method of treating a subject at risk of contracting influenza, comprising administering a suitable amount of a pharmaceutical composition of the disclosure is provided. In some embodiments, the method does not comprise administering an adjuvant to the subject.

### EXAMPLES

Below are examples of specific embodiments for carrying out the present disclosure. The examples are offered for illustrative purposes only and are not intended to limit the scope of the present disclosure in any way. Efforts have been made to ensure accuracy with respect to numbers used (e.g., amounts, temperatures, etc.), but some experimental error and deviation should, of course, be allowed for.

### Example 1. Design of Fusion Proteins and MIPs

This example describes the in silico design of a multi-epitope fusion protein comprising five or six peptides of the extracellular domains of the M2 protein (M2e) from Influenza A antigenic variants.

The average effectiveness of commercial influenza vaccines has been less than 50% over the last 13 years. Most of these vaccines are inactivated influenza vaccines (IIVs) made by purifying the influenza virus grown in chicken eggs, followed by splitting with detergent and subsequent chemical inactivation. The immune responses elicited by these commercially available vaccines are predominantly raised to the variable domains of viral hemagglutinin (HA), the major glycoprotein on the surface of the virus that changes from season to season (known as antigenic drift). Although these antibodies neutralize viral infections, they are typically strain-specific, and current vaccines are limited in their ability to display and present multiple antigenic strain variants. One or more new strains are typically introduced into the current seasonal vaccine each year in response to WHO recommendations, although sometimes despite the best efforts of all involved, the strain recommendations do not match circulating viruses. Given the poor performance of current influenza vaccines, there is a pressing need for improved or universal influenza vaccines.

M2 is a protein abundantly expressed on the surface of influenza-infected host cells and is an essential ion channel that enables the viral budding process. M2-specific antibodies inhibit viral replication by disrupting the viral budding process and targeting influenza-infected cells for killing by antibody-dependent cellular cytotoxicity (ADCC). The ectodomain of influenza A matrix protein 2 (M2e) is a target antigen for potential universal influenza A vaccines due to its remarkable conservation over time. To overcome its low immunogenicity, M2e requires a potent adjuvant, and as was demonstrated, peptide-adsorbed alum alone inadequately primes the immune response against influenza. Subsequently, a range of subunit fusion vaccines that combine M2e with carrier proteins, such as hepatitis B protein, keyhole limpet protein, and flagellin have been explored and developed, though many require supplementation by additional extrinsic adjuvants, such as incomplete Freund's adjuvant or MPL.

Since there is antigenic variability among the M2 ectodomains, multiple antigenic variants were used to help ensure specific immune responses are elicited to potentially all influenza strains based on the gene sequences of each of the known antigenic variants. It was found that the primary correlate of protective immunity is M2-specific IgG antibodies that are capable of providing protection when they are passively transferred to naïve animals.

Briefly, a universal influenza vaccine candidate was designed that contains multiple ectodomains of M2 antigens displayed in tandem as a ClyA fusion protein and is displayed on the surface of bacterial exosome-like vesicles (recombinant outer membrane vesicles, rOMVs).

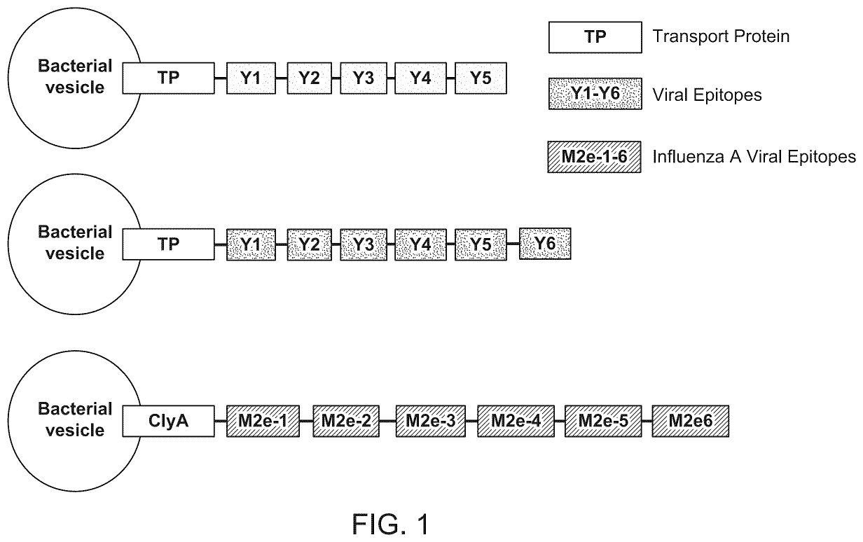

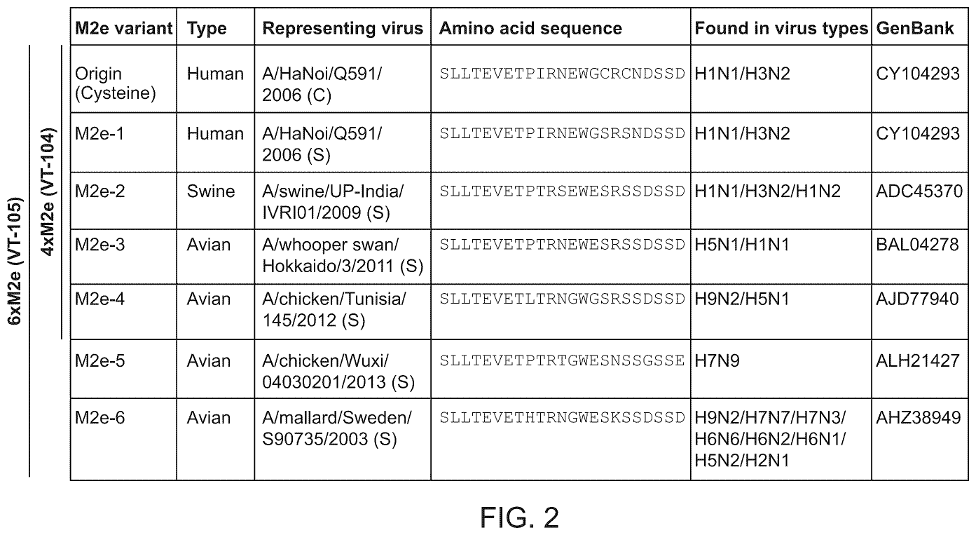

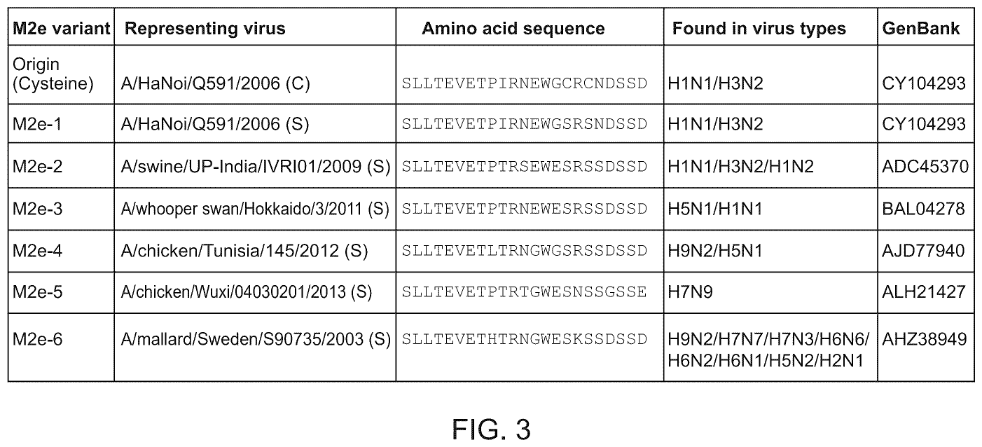

The designs included 6xM2e (VRT-105) and 4xM2e (VT-104). Exemplary designs are shown in FIG. 1. The details of the design and differences between 6xM2e (VRT-105) and 4xM2e (VT-104) are shown in FIG. 2. M2e variants, originating virus, and corresponding amino acid sequences are shown in FIG. 3. M2e variants, representing virus, a virus type is shown in Table 1, M2e sequences are shown in Table 2.

TABLE 1
M2e variants, representing virus, a virus type.

| M2e | | | |
|---|---|---|---|
| variants | Representing virus | Present in virus types | GenBank |
| M2e-1* | A/HaNoi/Q591/2006 (S) | H1N1/H3N2 | CY104293 |
| M2e-2* | A/swine/UP-India/IVRI01/2009 (S) | H1N1/H3N2/H1N2 | ADC45370 |
| M2e-3* | A/whooper swan/Hokkaido/3/2011 (S) | H5N1/H1N1 | BAL04278 |
| M2e-4* | A/chicken/Tunisia/145/2012 (S) | H9N2/H5N1 | AJD77940 |
| M2e-5* | A/chicken/Wuxi/04030201/2013 (S) | H7N9 | ALH21427 |
| M2e-6* | A/mallard/Sweden/S90735/2003 (S) | H9N2/H7N7/H7N3/H6N6/ | AHZ38949 |
| | | H6N2/H6N1/H5N2/H2N1 | |

TABLE 2
M2e Sequences

| SEQ | | |
|---|---|---|
| ID | | |
| NO: | Description | Sequence |
| 1 | ClyA | MTEIVADKTVEVVKNAIETADGA |
| | | LDLYNKYLDQVIPWQTFDETIKE |
| | | LSRFKQEYSQAASVLVGDIKTLL |
| | | MDSQDKYFEATQTVYEWCGVATQ |
| | | LLAAYILLFDEYNEKKASAQKDI |
| | | LIKVLDDGITKLNEAQKSLLVSS |
| | | QSFNNASGKLLALDSQLTNDFSE |
| | | KSSYFQSQVDKIRREAYAGAAAG |
| | | VVAGPFGLIISYSIAAAVVEGKL |
| | | IPELKNKLKSVQNFFTTLSNTVK |
| | | QANKDIDAAKLKLTTEIAAIGEI |
| | | KTETETTRFYVDYDDLMLSLLKE |
| | | AAKKMINTCNEYQKRHGKKTLFE |
| | | VPEV |
| 2 | Origin | SLLTEVETPIRNEWGCRCNDSSD |
| | (Cysteine) | |
| 3 | M2e-1 | SLLTEVETPIRNEWGSRSNDSSD |
| 4 | M2e-2 | SLLTEVETPTRSEWESRSSDSSD |
| 5 | M2e-3 | SLLTEVETPTRNEWESRSSDSSD |
| 6 | M2e-4 | SLLTEVETLTRNGWGSRSSDSSD |
| 7 | M2e-5 | SLLTEVETPTRTGWESNSSGSSE |
| 8 | M2e-6 | SLLTEVETHTRNGWESKSSDSSD |
| 9 | 6xM2e | GGGSGGGSLLTEVETPIRNEWGS |
| | | RSNDSSDGGGSGGGSLLTEVETP |
| | | TRSEWESRSSDSSDGGGSGGGSL |
| | | LTEVETPTRNEWESRSSDSSDGG |
| | | GSGGGSLLTEVETLTRNGWGSRS |
| | | SDSSDGGGSGGGSLLTEVETPTR |
| | | TGWESNSSGSSEGGGSGGGSLLT |
| | | EVETHTRNGWESKSSDSSD |

The in silico designed universal influenza vaccine candidate proteins M2e1x, M2e2x M2e3xM2e4xM2e5x, and M2e6x were probed for the degree of disorder in after protein folding using Phyre2, web portal for protein modeling, prediction and analysis. Kelly L A et al., Nature Protocols 10, 845-858 (2015).

Additionally, lipid tethering induces a native-like conformation of disordered epitopes and improves mAb binding.

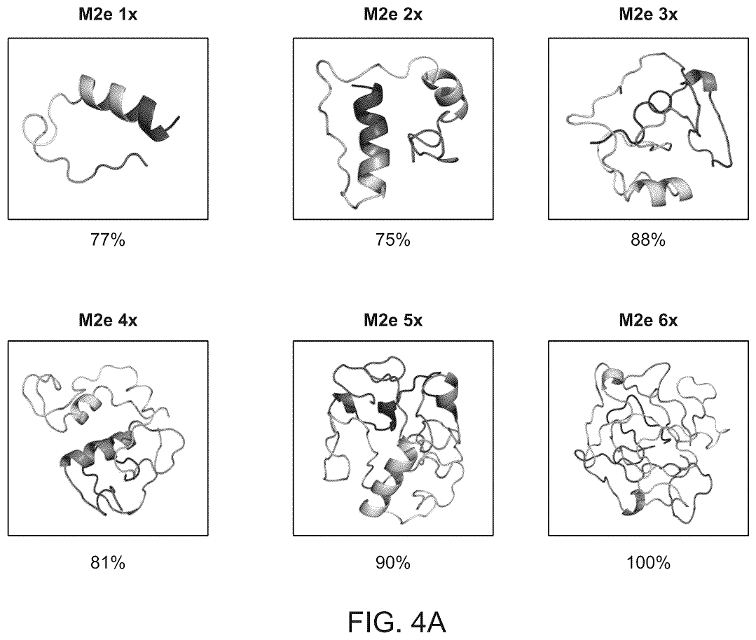

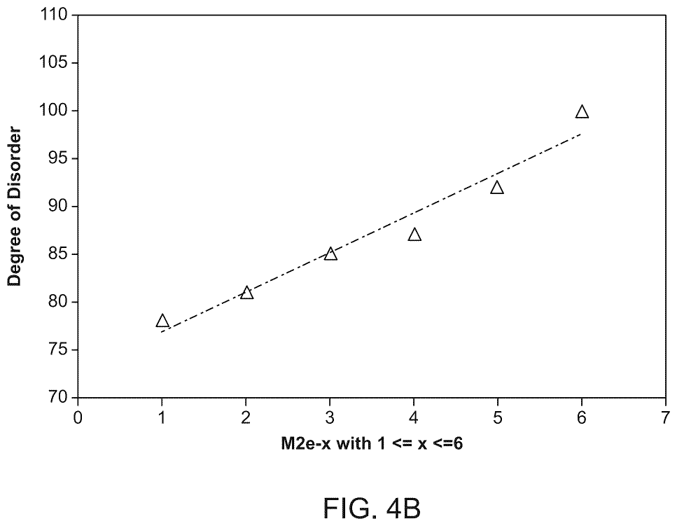

The results show, that predicted disorder increases with the length of the M2e fusion protein (FIGS. 4A and 4B).

### Example 2. Manufacture and Characterization of Expression Plasmid

### Construction of Expression Plasmid

ClyA and the four or six M2e sequences were linked to each other via flexible linkers.

DNA coding for ClyA-6xM2e (483 amino acids) was E. coli codon usage optimized and were cloned into a plasmid of 3689 bp.

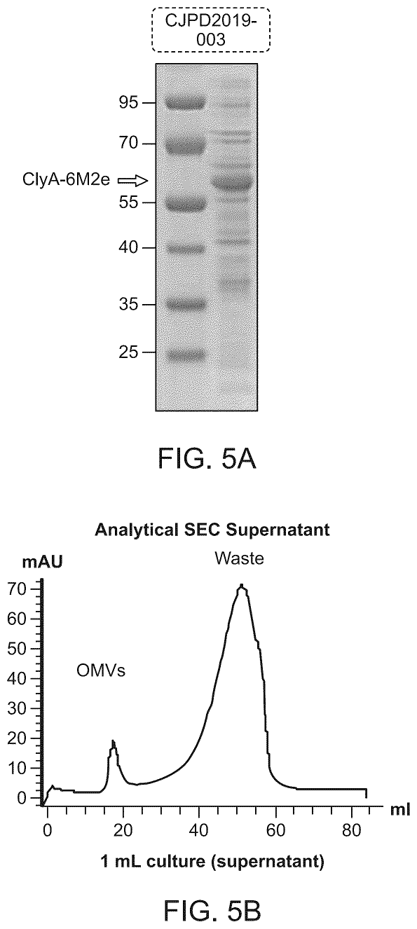

The custom plasmid was manufactured and characterized. The plasmid was transformed into E. coli DH10B and transformants selected on LB plates under kanamycin selection (50 μg/ml). Plasmids isolated from multiple isolates were subjected to restriction digest with specific restriction enzymes. Plasmids with the predicted agarose gel electrophoresis banding patterns were selected for DNA sequencing and clones sequenced to confirm the insert sequence. An exemplary SDS PAGE gel showing an expression of the ClyA-6M2e fusion protein is shown in FIG. 5A. Analysis of culture supernatant was performed by SDS-PAGE and analytical SEC FIG. 5B.

### Example 3. Manufacture and Characterization of E. coli Cell Bank

The rOMVs are derived from a commensal strain (Nissle 1917) of E. coli that is genetically engineered to hyper-vesiculate (overproduce rOMVs) and serve as both a vaccine delivery vehicle and an adjuvant for inducing a robust immunity without biologically active lipopolysaccharide (LPS). The commensal strain was selected because of its ability to induce more robust immune responses than other strains of E. coli. rOMV vaccines are nano-size lipid vesicles that can carry protein and polysaccharide antigens and are produced constitutively from the outer membrane and the periplasmic space of Gram negative bacteria. rOMVs have been shown to be stable (two years at 5° C. in solution), have the potential to be lyophilized for increased thermostability. Some rOMV vaccines are derived from a BSL-1 probiotic strain of E. coli (Nissle 1917) that is detoxified by structural remodeling of the lipopolysaccharide (LPS; Nissle-4 carries four acyl chains on its LPS instead of six acyl chains). This crippled form of endotoxin does not trigger a pyrogenic response in human blood nor activate cells through mouse or human Toll-like receptor-4 (TLR4); similar approaches have demonstrated an excellent safety profile.

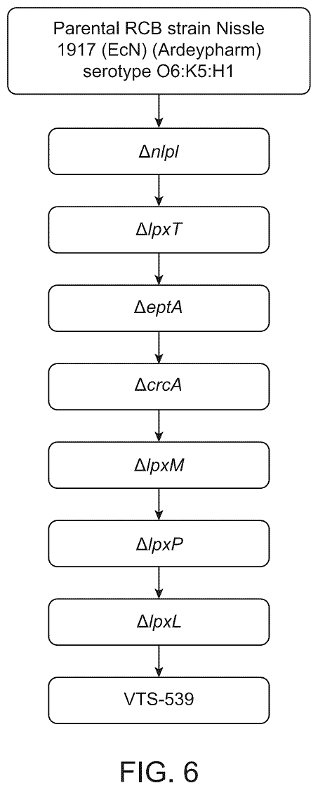

Briefly, the ClyA-6xM2e rOMVs were recombinantly expressed using an engineered derivative of the probiotic E. coli strain Nissle 1917 (EcN) serotype O6:K5:H1. Genetic engineering was performed to generate a cell line that (i) was deficient in the ability to synthesize pyrogenic forms of lipopolysaccharide (LPS) and (ii) that vesiculated (i.e. liberated rOMV) at a significantly higher rate than the parental strain. An overall description of the cell line genetic engineering is depicted in FIG. 6.

Knockouts were constructed in a two-step process. During the first step, the gene targeted for knockout was replaced by an antibiotic resistance cassette immediately flanked by tandem flippase recognition target (FRT) sites. Insertion clones were selected by antibiotics resistance and confirmed by PCR amplification of a unique gene locus reporting on a correct insertion. Following this, the antibiotic resistance cassette was removed by introduction and activation of a plasmid-encoded flippase (FLP) recombination, which recognizes the tandem FRT sites and excises the cassette between these sites. The FLP-encoding plasmid is temperature-sensitive and subsequently cured by culturing the cells at 37° C., a temperature that supports cell growth but not plasmid replication. Excision of the inserted cassette was confirmed by PCR amplification of the targeted gene locus. A “clean” deletion after cassette excision always results in a unique amplicon size compared with that from the wild-type and insertion mutation. Modifications are shown in Table 3.

TABLE 3
Modifications in E. coli strain Nissle.

| Modifi- | | |
|---|---|---|
| cation | Function | Specific effect |
| ΔlpxL, | LPS acylation | Deletion prevents addition of the #5 acyl chain |
| ΔlpxP | | |
| ΔlpxM | LPS acylation | Deletion prevents addition of the #6 acyl chain |
| ΔcrcA | LPS acylation | Deletion of conditionally active enzyme that |
| (pagP) | | adds acyl chains in response to certain |
| | | environmental stress |
| Δepta | LPS | Deletion prevents addition of an ethanolamine |
| | modification | group to lipid A. |
| ΔlpxT | LPS | Deletion prevents and addition of a phosphate |
| | modification | group to the lipid A |
| Δnlpl | Lipoprotein | Deletion induces hyper-vesiculation of OMV. |
| | modification | |

The strain was engineered to hyper-vesiculate and generate high yields of rOMV naturally, bypassing the need for detergent extraction used in other OMV vaccine preparations. Furthermore, heterologous protein antigens are targeted to the surface of the rOMVs using ClyA as a fusion and display protein. Together, this allows for diverse antigens to be displayed on the surface of the vesicle in a multimeric complex. rOMVs have an average diameter of 100 nm, which is ideal for efficient drainage into lymph nodes, uptake by antigen presenting cells, and induction of robust immune responses. The resulting rOMV vaccine candidates are non-toxic, well-tolerated and overcome poor immunogenicity of purified peptides in animal model studies.

Multiple epitopes can be displayed and arranged in a conformational manner that enables the induction of high levels of cross-reactive antibodies capable of providing cross-strain protection against influenza in mice and ferrets.

### Example 4. Animal Efficacy Studies of OMVs Expressing Multimeric M2e

VT-104 and VT-105 were tested for efficacy in animal studies for efficacy against influenza A strain.

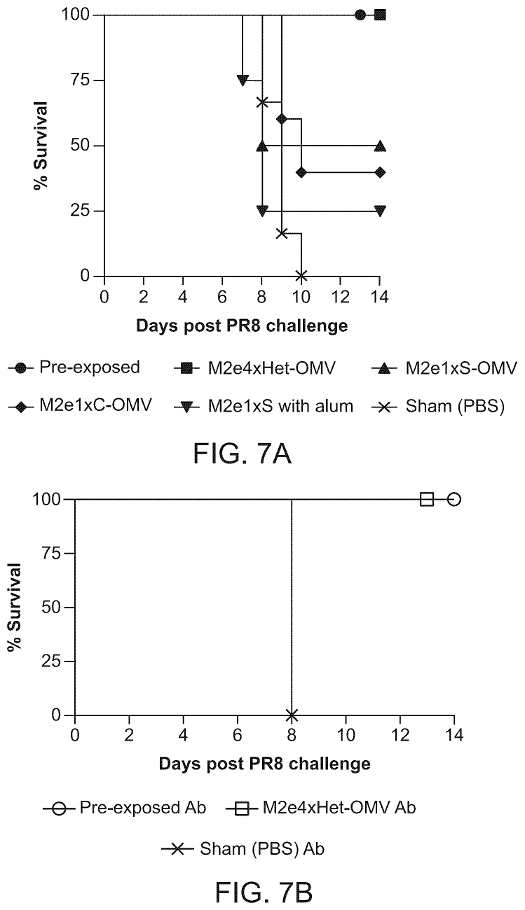

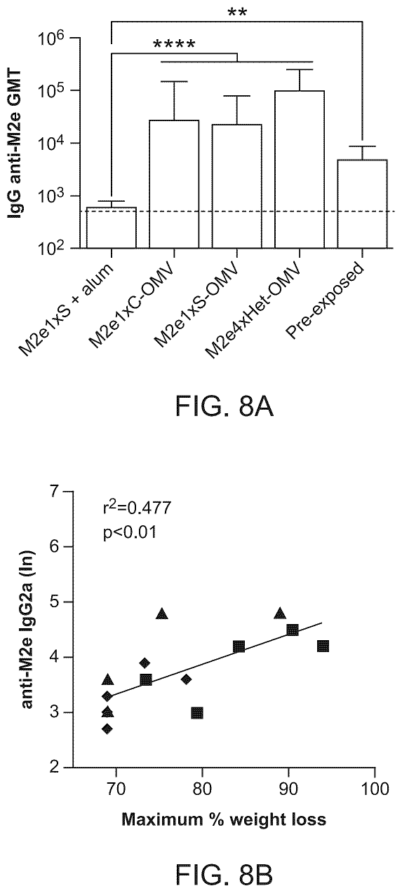

VT-104 was tested with an influenza A strain A/PR8/34 challenge in multiple mouse models. In each of the mouse model studies, the immunizations were performed as a prime/boost immunization regimen, four weeks apart containing 0.2 μg of total M2 antigen followed by viral challenge [24]. As shown in one of the studies all sham (PBS) control mice succumbed to infection by day 10 post-challenge, confirming a lethal challenge dose (FIGS. 7A and 8B). VT-104 vaccinated mice showed the least morbidity of the vaccinated groups. Survival analysis demonstrated that only M2e4xHet-OMVs (VT-104) provided significantly better protection than that of the sham (PBS) vaccine (p<0.01). In contrast, when M2e peptides were administered with alum, protection against lethal challenge was not achieved, with only 25% survival (¼) indicating the robustness of rOMV generated immune responses. Additionally, average lung and trachea viral titer of VT-104 vaccinated mice were approximately 10-fold lower than that of the sham (PBS) control at day 6 post challenge. Moreover, serum from mice immunized with the VT-104 provided 100% protection upon passive transfer to naïve mice.

It was found that by displaying M2e peptides as a ClyA fusion protein on the surface of rOMVs resulted in significantly elevated anti-M2e IgG2a titers versus IgG1 titers and achieved protection against influenza challenge without additional adjuvants (FIGS. 8A and 8B). Linear regression analysis of log transformed anti-M2e total IgG titers vs. the minimum percent original weight yielded a significant relationship (r2=0.403, p<0.05). The literature suggests IgG2a is a correlate of protection for M2e; therefore, IgG2a vs. lowest percent original weight was analyzed and found an even stronger relationship (r2=0.477, p<0.01), whereas IgG1 titers vs. lowest percent original weight did not have a significant relationship (r2=0.228, p>0.05). The proposed mechanism of protection for M2e-based vaccines is clearance of infected cells through antibody dependent cell cytotoxicity and phagocytosis (ADCC and ADCP) and it has been shown that IgG2a antibodies are both necessary and sufficient to provide protection in mice. The results add further support to this proposed mechanism. It was found that protection from weight loss during challenge most significantly correlated with IgG2a antibody levels. The minimum IgG2a antibody titer among passively immunized mice was 8,000, adding strength to the reported minimum protective IgG2a antibody titer of 10,000. Overall, the data support the premise that OMVs represent a promising adjuvant platform for development of an M2e-based universal influenza vaccine.

### Example 5. Efficacy Study of VT-104 and VT-105 in Ferret Challenged with H5N1

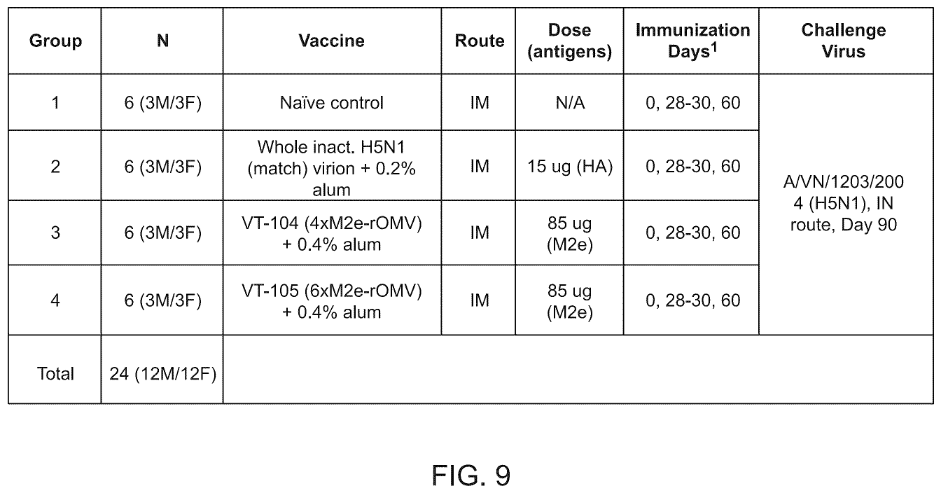

The efficacy of VT-105 was compared to VT-104 in the ferret H5N1 influenza model. For this study a total of 24 ferrets were divided into four groups containing 6 ferrets each for the study. Groups of ferrets were immunized with one of two rOMV-M2e vaccine candidates (M2 antigens, 85 μg), with the inactivated H5N1 vaccine (VN/1203/04) as a positive control, or with an empty rOMV serving as a negative control preparation. The ferrets were given three rounds of immunizations at 28-day intervals and blood samples collected to monitor generation of an antibody response. Thirty days after the last immunization, all ferrets were challenged with a lethal dose of the high path avian influenza virus (VN/1203/04; H5N1). Details of the study are shown in FIG. 9.

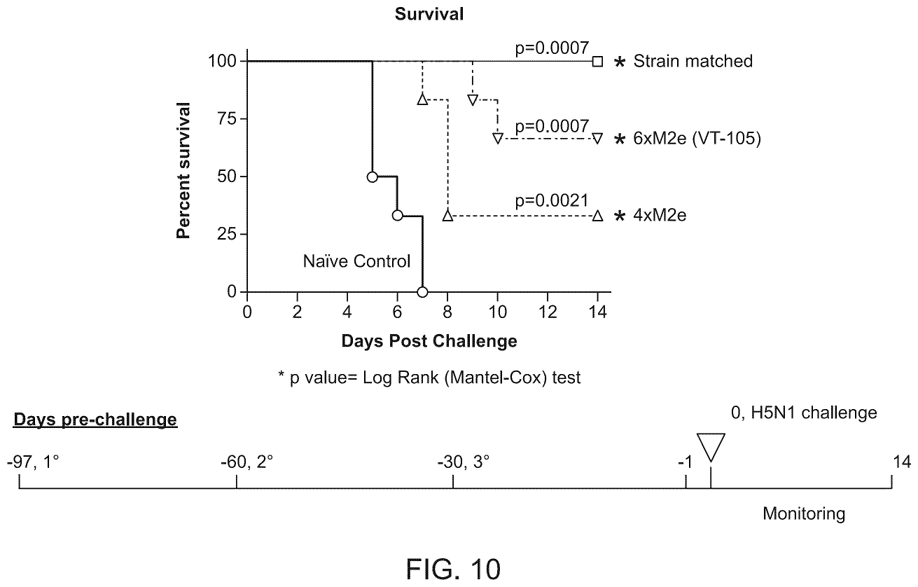

The results show that Infection with the H5N1 virus resulted in severe illness in the negative control ferrets (Group 1) and due to severe illness (extreme lethargy, neurological signs) none of the ferrets in this group survived the challenge and the remaining two ferrets were euthanized on Day 7 post challenge (FIG. 10). The ferrets in the positive (H5N1) control group had a favorable outcome and achieved 100% survival. The VT-104 group had a survival rate of 33% (2/6) whereas the VT-105 group fared better and achieved a 66% (4/6) survival rate.

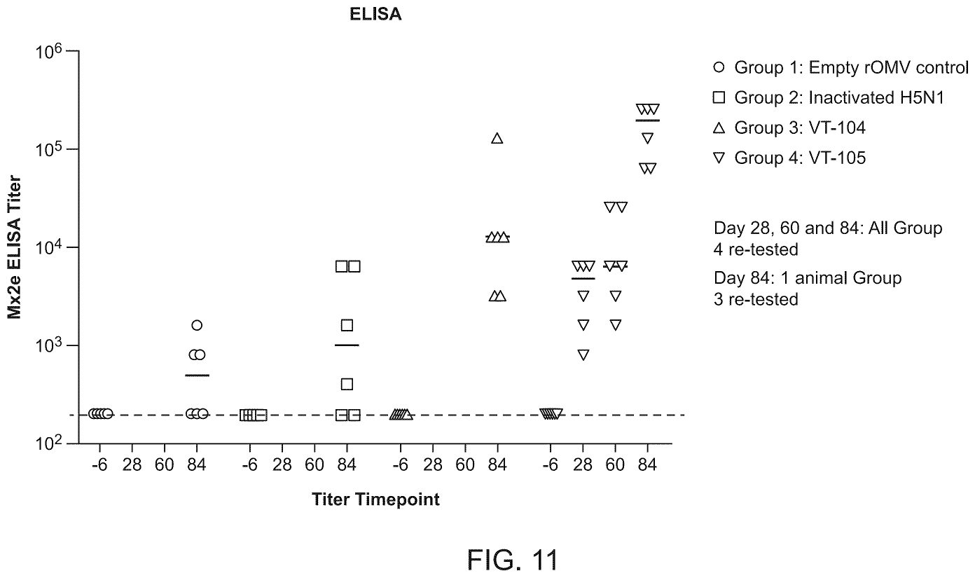

Antisera were evaluated in an M2e IgG ELISA for detection of influenza-specific antibodies. FIG. 11 depicts the results, showing the antibody titers in ferret sera during the 4 phases of the study period. As expected, all the pre-immunization sera were below the cut-off for the assay (200). Sera from all four study groups were tested at Day 84 (two weeks following the last immunization). At this time point, 3/6 ferrets in the negative control group had low levels of IgG titers (800-1,600) and the remaining three ferrets tested below level of detection. For the H5N1 inactivated vaccine group, 5/6 ferrets had low to modest IgG titers that ranged between 400 and 6,400. In contrast the two rOMV vaccine groups, which had received the M2e antigen, developed the highest titers. By Day 84, the highest responding group, VT-105 (6xM2e) had the most robust titers of 64,000-256,000.

While this invention has been particularly shown and described with references to preferred embodiments thereof, it will be understood by those skilled in the art that various changes in form and details may be made therein without departing from the scope of the invention encompassed by the appended claims.

SEQUENCE LISTING

| SEQ | | |
|---|---|---|
| ID | | |
| NO: | Description | Sequence |
| 1 | ClyA | MTEIVADKTVEVVKNAIETADGALDLYNKYLDQVIPWQTFDET |
| | | IKELSRFKQEYSQAASVLVGDIKTLLMDSQDKYFEATQTVYEWC |
| | | GVATQLLAAYILLFDEYNEKKASAQKDILIKVLDDGITKLNEAQ |
| | | KSLLVSSQSFNNASGKLLALDSQLTNDFSEKSSYFQSQVDKIRR |
| | | EAYAGAAAGVVAGPFGLIISYSIAAAVVEGKLIPELKNKLKSVQ |
| | | NFFTTLSNTVKQANKDIDAAKLKLTTEIAAIGEIKTETETTRFY |
| | | VDYDDLMLSLLKEAAKKMINTCNEYQKRHGKKTLFEVPEV |
| 2 | Origin | SLLTEVETPIRNEWGCRCNDSSD |
| | (Cysteine) | |
| 3 | M2e-1 | SLLTEVETPIRNEWGSRSNDSSD |
| 4 | M2e-2 | SLLTEVETPTRSEWESRSSDSSD |
| 5 | M2e-3 | SLLTEVETPTRNEWESRSSDSSD |
| 6 | M2e-4 | SLLTEVETLTRNGWGSRSSDSSD |
| 7 | M2e-5 | SLLTEVETPTRTGWESNSSGSSE |
| 8 | M2e-6 | SLLTEVETHTRNGWESKSSDSSD |
| 9 | 6xM2e | GGGSGGGSLLTEVETPIRNEWGSRSNDSSDGGGSGGGSLLTEVE |
| | | TPTRSEWESRSSDSSDGGGSGGGSLLTEVETPTRNEWESRSSDS |
| | | SDGGGSGGGSLLTEVETLTRNGWGSRSSDSSDGGGSGGGSLLTE |
| | | VETPTRTGWESNSSGSSEGGGSGGGSLLTEVETHTRNGWESKSS |
| | | DSSD |
| 10 | >sp\|P06821\|M2_13 | MSLLTEVETPIRNEWGCRCNGSSDPLAIAANIIGILHLILWILDR |
| | 4A1 Matrix protein | LFFKCIYRRFKYGLKGGPSTEGVPKSMREEYRKEQQSAVDADDGH |
| | 2 OS = Influenza A | FVSIELE |
| | virus (strain A/ | |
| | Puerto Rico/ | |
| | 8/1934 H1N1) | |
| 11 | M2e peptide | SLLTEVETPIRNEWGCRCNGSSD |

1. A multivalent influenza virus vaccine (MIV), said MIV comprising:
$$A - B - C$$
wherein
i) A is a transport protein or a fragment thereof comprising at least domains X₁ and X₂, wherein
a) X₁ is a transmembrane domain polypeptide, and
b) X₂ is a polypeptide capable of being operably linked to B;
ii) B is a multivalent immunogenic polypeptide (MIP) comprising at least five influenza virus immunogenic epitopes selected from the group consisting of Y₁, Y₂, Y₃, Y₄, Y₅, and Y₆,
iii) C is a membrane vesicle derived from a genetically modified gram positive bacteria;
wherein A and B are operably linked, resulting in a multivalent immunogenic transmembrane polypeptide;
wherein A-B is linked to C via the transmembrane domain X₁, thereby forming the MIV;
and wherein the MIV is capable of eliciting an immune response to at least two influenza strains when administered to a mammal.

2. A multivalent influenza virus vaccine (MIV), said MIV comprising:
a) a transport protein or a fragment thereof, wherein said transport protein comprises a transmembrane domain;
b) a multivalent immunogenic polypeptide (MIP) comprising at least five covalently linked influenza virus immunogenic epitopes;
c) a membrane vesicle derived from a genetically modified gram positive bacteria;
wherein the transport protein of a) is operably linked to the MIP of b), resulting in a multivalent immunogenic transmembrane polypeptide;
wherein the multivalent immunogenic transmembrane polypeptide is linked to the vesicle forming the MIV; and
wherein the MIV is capable of eliciting an immune response to at least 2 influenza virus strains when administered to a mammal.

3. The MIV of claim 1, wherein the transport protein is an adhesin, immunomodulatory compound, protease, or toxin, or a fragment thereof.

4. The MIV of claim 3, wherein the transport protein is ClyA.

5. The MIV of claim 4, wherein the ClyA comprises an amino acid sequence at least about 80% identical to SEQ ID NO: 1.

6. The MIV of claim 1, wherein the at least five influenza virus immunogenic epitopes are fused in tandem.

7. The MIV of claim 1, wherein the operable linkage between A and B comprises a covalent linkage.

8. The MIV of claim 1, wherein the at least five influenza virus immunogenic epitopes are fused to a N- or C-terminus of the transport protein.

9. The MIV of claim 1, wherein the at least five influenza virus immunogenic epitopes are presented on outside of the vesicle.

10. The MIV of claim 1, wherein the influenza virus is influenza A.

11. The MIV of claim 10, wherein the influenza A is human, swine, or avian.

12. The MIV of claim 11, wherein the avian is a chicken, a whooper a swan, a quail, or a mallard.

13. The MIV of claim 10, wherein the influenza A is influenza subtype is H1, H2, H3, H5, H6, H7, or H9.

14. The MIV of claim 10, wherein the influenza virus is selected from the group consisting of H1N1, H1N2, H2N1, H3N2, H5N1, H5N2, H9N2, H7N9, H7N7, H7N3, H6N6, H6N2, and H6N1.

15-16. (canceled)

17. The MIV of claim 1, wherein at least one of the at least five influenza virus immunogenic epitopes comprises a consensus sequence or a non-naturally occurring sequence.

18. The MIV of claim 1, wherein the MIP comprises an influenza A matrix protein 2 extracellular (M2e) peptide or fragment thereof.

19. The MIV of claim 17, wherein the MIP comprises five M2e peptides.

20-27. (canceled)

28. The MIV of claim 1, wherein Y₁, Y₂, Y₃, Y₄, Y₅, and Y₆ are selected from the group consisting of a human M2e peptide, a swine M2e peptide, a swan M2e peptide, a chicken M2e peptide, and a mallard M2e peptide.

29. (canceled)

30. The MIV of claim 18, wherein the multivalent immunogenic transmembrane polypeptide comprises the amino acid sequence having a formula ClyA-(M2e)₆.

31. The MIV of claim 18, wherein the multivalent immunogenic transmembrane polypeptide comprises the amino acid sequence having a formula ClyA-(M2e-1)-(M2e-2)-(M2e-3)-(M2e-4)-(M2e-5)-(M2e-6), wherein M2e-1 comprises human M2e peptide, wherein M2e-2 comprises swine M2e peptide, wherein M2e-3 comprises swan M2e peptide, wherein M2e-4 comprises chicken M2e peptide, wherein M2e-5 comprises chicken M2e peptide, and wherein M2e-6 comprises mallard M2e peptide.

32. The MIV of claim 18, wherein the multivalent immunogenic transmembrane polypeptide comprises the amino acid sequence having a formula ClyA-(M2e-1)-(M2e-2)-(M2e-3)-(M2e-4)-(M2e-5)-(M2e-6), wherein M2e-1 comprises the amino acid sequence at least about 90% identical to SEQ ID NO: 3, wherein M2e-2 comprises the amino acid sequence at least about 90% identical to SEQ ID NO: 4, wherein M2e-3 comprises the amino acid sequence at least about 90% identical to SEQ ID NO: 5, wherein M2e-4 comprises the amino acid sequence at least about 90% identical to SEQ ID NO: 6, wherein M2e-5 comprises the amino acid sequence at least about 90% identical to SEQ ID NO: 7, and wherein M2e-6 comprises the amino acid sequence at least about 90% identical to SEQ ID NO: 8.

33. The MIV of claim 1, wherein the multivalent immunogenic transmembrane polypeptide comprises the amino acid sequence at least about 80% identical to SEQ ID NO: 9.

34-65. (canceled)


SEQUENCE LISTING (22 sequences)

SEQ ID NO: 1 - protein, 303 aa, synthetic construct
MTEIVADKTVEVVKNAIETADGALDLYNKYLDQVIPWQTFDETIKELSRFKQEYSQAASVLVGDIKTLLMDSQDKYFEATQTVYEWCGVATQLLAAYILLFDEYNEKKASAQKDILIKVLDDGITKLNEAQKSLLVSSQSFNNASGKLLALDSQLTNDFSEKSSYFQSQVDKIRREAYAGAAAGVVAGPFGLIISYSIAAAVVEGKLIPELKNKLKSVQNFFTTLSNTVKQANKDIDAAKLKLTTEIAAIGEIKTETETTRFYVDYDDLMLSLLKEAAKKMINTCNEYQKRHGKKTLFEVPEV

SEQ ID NO: 2 - protein, 23 aa, synthetic construct
SLLTEVETPIRNEWGCRCNDSSD

SEQ ID NO: 3 - protein, 23 aa, synthetic construct
SLLTEVETPIRNEWGSRSNDSSD

SEQ ID NO: 4 - protein, 23 aa, synthetic construct
SLLTEVETPTRSEWESRSSDSSD

SEQ ID NO: 5 - protein, 23 aa, synthetic construct
SLLTEVETPTRNEWESRSSDSSD

SEQ ID NO: 6 - protein, 23 aa, synthetic construct
SLLTEVETLTRNGWGSRSSDSSD

SEQ ID NO: 7 - protein, 23 aa, synthetic construct
SLLTEVETPTRTGWESNSSGSSE

SEQ ID NO: 8 - protein, 23 aa, synthetic construct
SLLTEVETHTRNGWESKSSDSSD

SEQ ID NO: 9 - protein, 180 aa, synthetic construct
GGGSGGGSLLTEVETPIRNEWGSRSNDSSDGGGSGGGSLLTEVETPTRSEWESRSSDSSDGGGSGGGSLLTEVETPTRNEWESRSSDSSDGGGSGGGSLLTEVETLTRNGWGSRSSDSSDGGGSGGGSLLTEVETPTRTGWESNSSGSSEGGGSGGGSLLTEVETHTRNGWESKSSDSSD

SEQ ID NO: 10 - protein, 97 aa, Influenza A virus
MSLLTEVETPIRNEWGCRCNGSSDPLAIAANIIGILHLILWILDRLFFKCIYRRFKYGLKGGPSTEGVPKSMREEYRKEQQSAVDADDGHFVSIELE

SEQ ID NO: 11 - protein, 23 aa, synthetic construct
SLLTEVETPIRNEWGCRCNGSSD

SEQ ID NO: 12 - protein, 40 aa, synthetic construct
  VARIANT 3..40: note = GS repeats may be deleted
GSGSGSGSGSGSGSGSGSGSGSGSGSGSGSGSGSGSGSGS

SEQ ID NO: 13 - protein, 60 aa, synthetic construct
  VARIANT 4..60: note = GGS repeats may be deleted
GGSGGSGGSGGSGGSGGSGGSGGSGGSGGSGGSGGSGGSGGSGGSGGSGGSGGSGGSGGS

SEQ ID NO: 14 - protein, 80 aa, synthetic construct
  VARIANT 5..80: note = GGGS repeats may be deleted
GGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 15 - protein, 100 aa, synthetic construct
  VARIANT 6..100: note = GGGGS repeats may be deleted
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 16 - protein, 20 aa, synthetic construct
  VARIANT 2..20: note = Residue may be deleted
GGGGGGGGGGGGGGGGGGGG

SEQ ID NO: 17 - protein, 36 aa, synthetic construct
  VARIANT 7..36: note = GGSGGD repeats may be deleted
GGSGGDGGSGGDGGSGGDGGSGGDGGSGGDGGSGGD

SEQ ID NO: 18 - protein, 36 aa, synthetic construct
  VARIANT 7..36: note = GGSGGE repeats may be deleted
GGSGGEGGSGGEGGSGGEGGSGGEGGSGGEGGSGGE

SEQ ID NO: 19 - protein, 21 aa, synthetic construct
  VARIANT 8..21: note = GGGSGGG repeats may be deleted
GGGSGGGGGGSGGGGGGSGGG

SEQ ID NO: 20 - protein, 33 aa, synthetic construct
  VARIANT 12..33: note = GGGSGSGGGGS repeats may be deleted
GGGSGSGGGGSGGGSGSGGGGSGGGSGSGGGGS

SEQ ID NO: 21 - protein, 33 aa, synthetic construct
  VARIANT 12..33: note = GGGGGPGGGGP repeats may be deleted
GGGGGPGGGGPGGGGGPGGGGPGGGGGPGGGGP

SEQ ID NO: 22 - protein, 7 aa, synthetic construct
GGGSGGG